(19) United States
(12) Patent Application Publication
(10) Pub. No.: US 2025/0032002 A1
(43) Pub. Date: Jan. 30, 2025

(54) WEARABLE DEVICE AND OPERATING METHOD THEREOF

(71) Applicant: Samsung Electronics Co., Ltd., Suwon-si (KR)

(72) Inventors: Pyeonggook JUNG, Suwon-si (KR); Jungpil YU, Suwon-si (KR)

(21) Appl. No.: 18/787,241

(22) Filed: Jul. 29, 2024

Related U.S. Application Data
(63) Continuation of application No. PCT/KR2024/006393, filed on May 10, 2024.

(30) Foreign Application Priority Data
Jul. 24, 2023 (KR) 10-2023-0096033

(51) Int. Cl. A61B 5/11; G06F 1/16

(52) U.S. Cl. CPC A61B 5/1121; G06F 1/163

(57) ABSTRACT

A wearable device may measure a first joint angle of a user, measure a second joint angle of the user, generate a filter input based on a difference between first angle data obtained by measuring the first joint angle and second angle data obtained by measuring the second joint angle, perform filtering on the generated filter input through a filter so that a change timepoint of a torque rotation direction of a driving module of the wearable device corresponds to a peak timepoint of the generated filter input by delaying a phase of the generated filter input through the filter, generate a torque based on a filter output generated by the filtering, and provide the generated torque to the user.

### CROSS-REFERENCE TO RELATED APPLICATIONS

This application is a continuation application of International Application No. PCT/KR2024/006393 designating the United States, filed on May 10, 2024, in the Korean Intellectual Property Receiving Office, and claiming priority to Korean Patent Application No. 10-2023-0096033, filed on Jul. 24, 2023, in the Korean Intellectual Property Office, the disclosures of which are all hereby incorporated by reference herein in their entireties.

### BACKGROUND

### 1. Field

Example embodiments relate to a wearable device and/or an operating method thereof.

### 2. Description of Related Art

In general, a walking assistance device refers to a mechanism or device that helps a person to exercise and/or which helps a patient, who cannot walk on his own due to various diseases, accidents, and the like, to perform walking exercises for rehabilitation treatment. With the recent intensifying aging societies, a growing number of people experience inconvenience in walking or have difficulty in normal walking due to malfunctioning joint issues, and there is increasing interest in walking assistance devices. A walking assistance device may be worn on a body of a user to assist the user with exercise, and/or with walking by providing a necessary or desired muscular strength and to induce the user to walk in a normal walking pattern.

### SUMMARY

According to an example embodiment, a wearable device may include a driving module, comprising a motor and/or circuitry, configured to generate a torque and provide the generated torque to the user, a first sensor configured to measure a first joint angle of the user, a second sensor configured to measure a second joint angle of the user, and at least one processor comprising processing circuitry. The at least one processor may individually and/or collectively generate a filter input to be filtered by a filter based on a difference between first angle data obtained by measuring the first joint angle by the first sensor and second angle data obtained by measuring the second joint angle by the second sensor. The at least one processor may individually and/or collectively perform filtering on the generated filter input through the filter so that a change timepoint of a torque rotation direction of the driving module may correspond to a peak timepoint of the generated filter input by delaying a phase of the generated filter input through the filter. The at least one processor may individually and/or collectively control the driving module based on a filter output generated by the filtering, so that a torque based on the generated filter output may be generated by the driving module.

According to an example embodiment, a wearable device may include a driving module configured to generate a torque and provide the generated torque to the user, one or more sensors configured to obtain motion information of a user by sensing a motion of the user, and at least one processor comprising processing circuitry. The at least one processor may individually and/or collectively generate a filter input based on the obtained motion information. The at least one processor may individually and/or collectively perform filtering on the generated filter input through a filter so that a change timepoint of a torque rotation direction of the driving module may correspond to a peak timepoint of the generated filter input by delaying a phase of the generated filter input through the filter. The at least one processor may individually and/or collectively control the driving module based on a filter output generated by the filtering, so that a torque based on the generated filter output may be generated by the driving module.

According to an example embodiment, an operating method of a wearable device may include measuring a first joint angle of a user and measuring a second joint angle of the user. The operating method of the wearable device may include generating a filter input based on a difference between first angle data obtained by measuring the first joint angle and second angle data obtained by measuring the second joint angle. The operating method of the wearable device may include performing filtering on the generated filter input through a filter so that a change timepoint of a torque rotation direction of a driving module of the wearable device may correspond to a peak timepoint of the generated filter input by delaying a phase of the generated filter input through the filter. The operating method of the wearable device may include generating a torque based on a filter output generated by the filtering. The operating method of the wearable device may include providing the generated torque to the user.

### BRIEF DESCRIPTION OF THE DRAWINGS

The above and other aspects, features, and advantages of certain example embodiments of the present disclosure will be more apparent from the following detailed description, taken in conjunction with the accompanying drawings, in which:

FIG. 1A is a diagram illustrating an overview of a wearable device worn on a body of a user according to an example embodiment;

FIG. 1B is a diagram illustrating an example of a system including a wearable device according to an example embodiment;

FIG. 2A is a rear schematic view of a wearable device according to an example embodiment;

FIG. 2B is a left side view of a wearable device according to an example embodiment;

FIGS. 3A and 3B are block diagrams illustrating examples of a configuration of a wearable device according to an example embodiment;

FIG. 4 is a diagram illustrating an interaction between a wearable device and an electronic device according to an example embodiment;

FIG. 5 is a diagram illustrating an example of a configuration of an electronic device according to an example embodiment;

FIG. 6 is a diagram illustrating an example of motion information of a user obtained by a wearable device according to an example embodiment;

FIG. 7 is a flowchart illustrating an example of an operating method of a wearable device according to an example embodiment;

FIG. 8 is a flowchart illustrating an example of an operating method of a wearable device according to an example embodiment;

FIGS. 9, 10, and 11 are diagrams illustrating examples of filtering by a wearable device according to an example embodiment;

FIGS. 12 and 13 are diagrams illustrating examples of a signal processing operation of a wearable device according to an example embodiment;

FIGS. 14 and 15 are diagrams illustrating examples of an operation of a wearable device according to an example embodiment;

FIG. 16 is a diagram illustrating an example of an operation of a wearable device according to an example embodiment; and

FIG. 17 is a flowchart illustrating an example of an operating method of a wearable device according to an example embodiment.

### DETAILED DESCRIPTION

The following detailed structural or functional description is provided as an example only and various alterations and modifications may be made to the embodiments. Here, the embodiments are not construed as limited to the disclosure and should be understood to include all changes, equivalents, and replacements within the idea and the technical scope of the disclosure.

Terms, such as first, second, and the like, may be used herein to describe components. Each of these terminologies is not used to define an essence, order or sequence of a corresponding component but used merely to distinguish the corresponding component from other component(s). For example, a first component may be referred to as a second component, and similarly the second component may also be referred to as the first component. It should be noted that if it is described that one component is “connected”, “coupled”, or “joined” to another component, at least a third component(s) may be “connected”, “coupled”, and “joined” between the first and second components, although the first component may be directly connected, coupled, or joined to the second component.

As used herein, the singular forms “a”, “an”, and “the” include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, embodiments will be described in detail with reference to the accompanying drawings. When describing the embodiments with reference to the accompanying drawings, like reference numerals refer to like elements and any repeated description related thereto will be omitted.

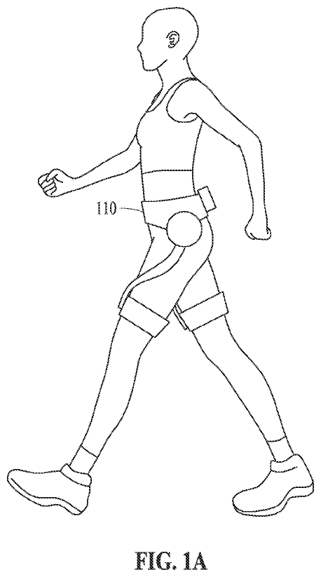

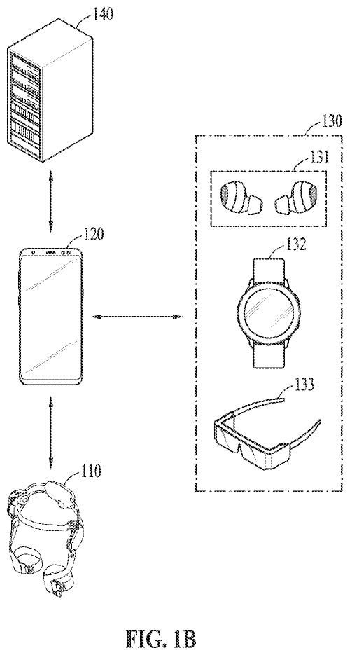

FIG. 1A is a diagram illustrating an overview of a wearable device worn on a body of a user according to an embodiment.

Referring to FIG. 1, a wearable device 110 may be a device worn on a body of a user to assist the user in walking, exercising, and/or working. In embodiments, the term “wearable device” may be replaced with “wearable robot,” “walking assistance device,” or “exercise assistance device”. The user may be a human or an animal, but is not limited thereto. The wearable device 110 may be worn on a body (e.g., a lower body (the legs, ankles, knees, etc.), an upper body (the torso, arms, wrists, etc.), or the waist) of the user to provide an external force such as an assistance force and/or a resistance force to a body motion of the user. The assistance force may be a force applied in the same direction as the body motion direction of the user, and the resistance force may be a force applied in a direction opposite to the body motion direction of the user. The term “resistance force” may also be referred to as “exercise load”.

When the wearable device 110 performs a walking assist function to assist the user in walking, the wearable device 110 may assist a portion or entirety of a leg of the user by providing an assistance force to the body of the user, thereby assisting the user in walking. The wearable device 110 may enable the user to walk independently or to walk for a long time by providing a force required for the user to walk, thereby extending the walking ability of the user. The wearable device 110 may help in improving an abnormal walking habit or gait posture of a walker.

When the wearable device 110 performs an exercise function to enhance the exercise effect of the user, the wearable device 110 may hinder a body motion of the user or provide resistance to a body motion of the user by providing a resistance force to the body of the user. When the wearable device 110 is, for example, a hip-type wearable device, the wearable device 110 may provide an exercise load to a body motion of the user while being worn on the legs, thereby enhancing the exercise effect of the user. The user may perform a walking motion while wearing the wearable device 110 for exercise. In this case, the wearable device 110 may apply a resistance force to the leg motion during the walking motion of the user.

In various embodiments, an example of a hip-type wearable device 110 that is worn on the waist and legs is described for ease of description. However, as described above, the wearable device 110 may be worn on another body part (e.g., the upper arms, lower arms, hands, calves, and feet) other than the waist and legs (particularly, the thighs), and the shape and configuration of the wearable device 110 may vary depending on the body part on which the wearable device 110 is worn.

FIG. 1B is a diagram illustrating an example of a system including a wearable device according to an embodiment.

Referring to FIG. 1B, an electronic device 120 may communicate with the wearable device 110 and remotely control the wearable device 110. The electronic device 120 may be various types of devices. The electronic device 120 may include, for example, a portable communication device (e.g., a smart phone), a computer device, a portable multimedia device, or a home appliance, but is not limited thereto.

According to an embodiment, the electronic device 120 and/or the wearable device 110 may be connected to another wearable device 130. For example, the wearable device 110, the electronic device 120, and the other wearable device 130 may be connected to each other through a wireless communication link (e.g., a Bluetooth communication link). The other wearable device 130 may include, for example, wireless earphones 131, a smart watch 132, or smart glasses 133, but is not limited thereto. The smart watch 132 may be a watch-type wearable device (or a watch-type electronic device), and the smart glasses 133 may be an eyewear-type wearable device (or an eyewear-type electronic device).

In an embodiment, the smart watch 132 may control the wearable device 110. When the smart watch 132 is connected to the electronic device 120 through a wireless communication link, and the electronic device 120 is connected to the wearable device 110 through a wireless communication link, the smart watch 132 may control the wearable device 110 through the electronic device 120. Embodiments are not limited thereto, and the smart watch 132 may be directly connected to the wearable device 110 and control the wearable device 110.

In an embodiment, the electronic device 120 may transmit, to the other wearable device 130, a control signal to instruct to provide a user with feedback corresponding to a state of the wearable device 110. The other wearable device 130 may provide (or output) feedback (e.g., at least one of visual feedback, auditory feedback, or tactile feedback) corresponding to the state of the wearable device 110 in response to the reception of the control signal.

In an embodiment, the electronic device 120 may communicate with a server 140 using short-range wireless communication (e.g., Wi-Fi) or mobile communication (e.g., 4G, 5G, etc.).

In an embodiment, the electronic device 120 may receive profile information of the user from the user. The profile information may include, for example, at least one of the age, gender, height, weight, or body mass index (BMI), or a combination thereof. The electronic device 120 may transmit the profile information of the user to the server 140.

In an embodiment, the electronic device 120 and/or the wearable device 110 may request the user to perform one or more target motions to determine (or check) the exercise ability of the user. The one or more target motions may include, for example, a knee lift, a backward leg stretch, etc. The knee lift may be an exercise (or a motion) that starts from a position of the user standing straight with two feet on the ground and returns to the standing position after lifting a leg backward as much as possible without bending at the waist. The backward leg stretch may be an exercise (or a motion) that starts from a position of the user standing straight with the hands on the wall and returns to the standing position after lifting a leg backward as much as possible without bending at the waist.

In an embodiment, the wearable device 110 may obtain motion information of the user performing a target motion using a sensor (e.g., an inertial measurement unit (IMU)), and transmit the obtained motion information to the electronic device 120. The electronic device 120 may transmit the obtained motion information to the server 140.

In an embodiment, the server 140 may determine a target amount of exercise of the user for each of the exercise types (e.g., strength training, balance exercise, and aerobic exercise) through the profile information and motion information received from the electronic device 120. The server 140 may transmit the target amount of exercise for each exercise type to the electronic device 120.

In an embodiment, the server 140 may include a database in which information about a plurality of exercise programs to be provided to the user is stored. For example, the server 140 may manage a user account of the user of the electronic device 120 or the wearable device 110. The server 140 may store and manage a workout program performed by the user and a result of performance with respect to the workout program in link with the user account.

In an embodiment, at least one of the wearable device 110, the electronic device 120, or the server 140 may provide the user with various exercise programs to achieve an exercise goal in various exercise environments desired by the user. The exercise goal may include, for example, at least one of muscle strength improvement, physical strength improvement, cardiovascular endurance improvement, core stability improvement, flexibility improvement, or symmetry improvement, or a combination thereof.

In an embodiment, at least one of the wearable device 110, the electronic device 120, or the server 140 may recommend exercise programs to the user to achieve the exercise goal of the user. Each exercise program may include one or more exercise types (e.g., running, a lunge, etc.) to achieve an exercise goal. For example, running may be an exercise type for improving the cardiovascular endurance of the user. For example, a lunge may be an exercise type for improving the core stability of the user. A combination of exercise types forming each exercise program may vary according to the exercise goal of the user. The electronic device 120 may provide the user with various exercise programs according to the combination of the plurality of exercise types, even for the same exercise goal.

In an embodiment, the plurality of exercise types may be stored in at least one of the wearable device 110, the electronic device 120, or the server 140 as a database. At least one of the wearable device 110, the electronic device 120, or the server 140 may generate the plurality of exercise programs based on a variety of information about the user and recommend a target exercise program among the plurality of exercise programs to the user in consideration of the exercise goal or an exercise performance state of the user. For example, at least one of the wearable device 110, the electronic device 120, or the server 140 may determine the target exercise program to recommend to the user based on at least one of the exercise goal, an exercise history, or an exercise performance result of the user. Accordingly, a new exercise program may be recommended to the user even if the user performs an exercise every day under the same exercise goal, and the user may feel like performing a different exercise from the previous exercise by performing the new exercise program.

In an embodiment, the server 140 may store exercise history information of the user. The exercise history information may include, for example, an evaluation result (e.g., an exercise duration, a walking symmetry, etc.) of exercises that the user has previously performed while wearing the wearable device 110. The server 140 may receive the evaluation result of the exercises from the wearable device 110 (or the electronic device 120) and store the evaluation result of the exercises.

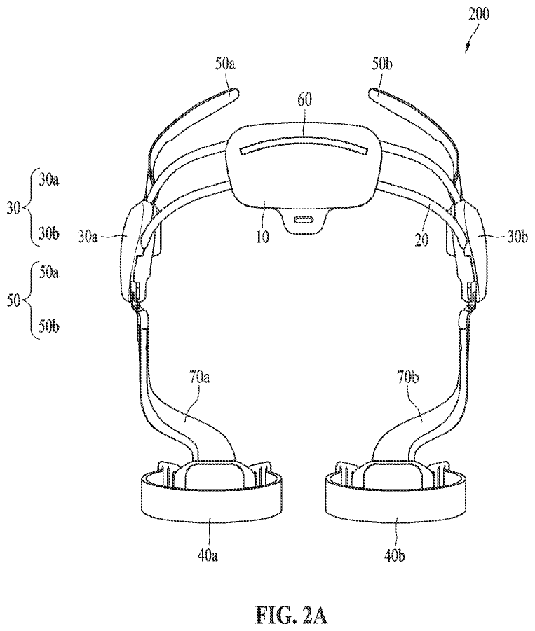

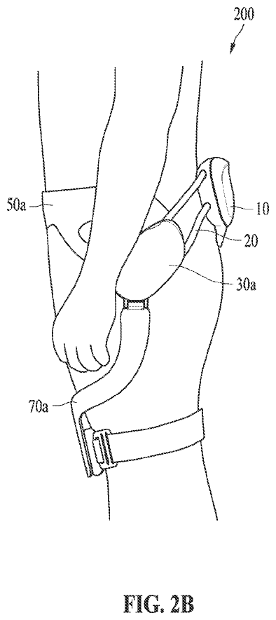

FIG. 2A is a rear schematic view of a wearable device according to an embodiment. FIG. 2B is a left side view of a wearable device according to an embodiment.

A wearable device 200 shown in FIGS. 2A and 2B may be an example of the wearable device 110.

Referring to FIG. 2A, according to an embodiment, the wearable device 200 may include a base body 10, a base frame 20, a driving module 30, thigh fastening portions 40a and 40b, a main belt 50, and leg driving frames 70a and 70b. Each “driving module” herein may comprise a motor and/or circuitry.

According to an embodiment, the base body 10 may be positioned on the lumbar region (an area of the lower back) of the user while the user is wearing the wearable device 200. The base body 10 may be mounted on the lumbar region of the user to provide a cushioning feeling to the lower back of the user and may support the lower back of the user. The base body 10 may be hung on the hip region (an area of the hips) of the user to prevent or reduce chances of the wearable device 200 from being separated downward due to gravity while the user is wearing the wearable device 200. The base body 10 may distribute a portion of the weight of the wearable device 200 to the lower back of the user while the user is wearing the wearable device 200. The base body 10 may be connected, directly or indirectly, to the base frame 20. Base frame connecting elements (not shown) to be connected to the base frame 20 may be provided at both end portions of the base body 10.

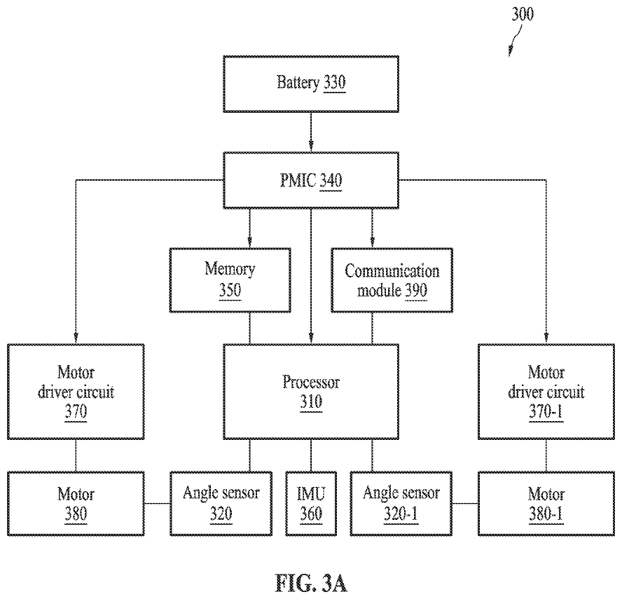

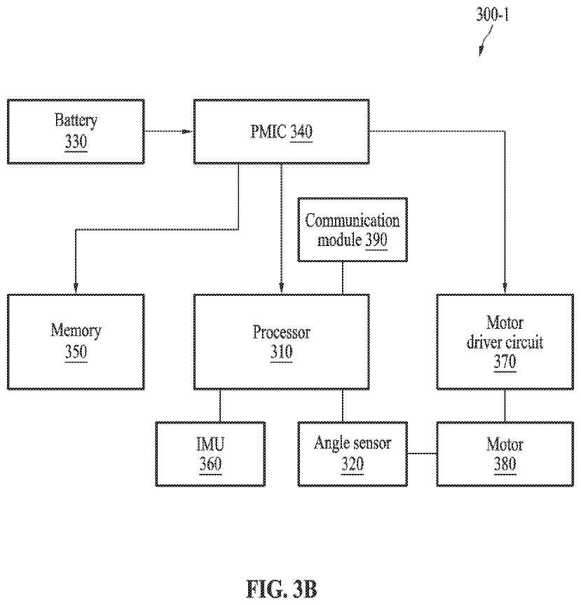

According to an embodiment, the base body 10 may include a lighting unit 60. The lighting unit 60 may include a plurality of light sources (e.g., light-emitting diodes (LEDs)). The lighting unit 60 may emit light by control of a processor (e.g., a processor 310 of FIGS. 3A and 3B described below). According to embodiments, the processor may control the lighting unit 60 such that visual feedback corresponding to the state of the wearable device 200 (e.g., a booting state, a sensing state, etc.) may be provided (or output) to the user through the lighting unit 60.

According to an embodiment, the base frame 20 may extend from both end portions of the base body 10. The lumbar region of the user may be accommodated inside the base frame 20. The base frame 20 may include at least one rigid body beam. Each beam may be in a curved shape having a preset curvature to enclose the lumbar region of the user. The main belt 50 may be connected, directly or indirectly, to an end portion of the base frame 20. The driving module 30 may be mounted on the base frame 20. The base frame 20 may include a connector (not shown) for mounting the driving module 30 thereon.

According to an embodiment, the driving module 30 may include a first driving module 30a positioned on the left side of the user while the user is wearing the wearable device 200, and a second driving module 30b positioned on the right side of the user while the user is wearing the wearable device 200.

According to an embodiment, the first driving module 30a may include a first angle sensor (e.g., a first encoder or a first Hall sensor) for measuring the left hip joint angle of the user. The second driving module 30b may include a second angle sensor (e.g., a second encoder or a second Hall sensor) for measuring the right hip joint angle of the user.

According to an embodiment, the first driving module 30a may include a first actuator and a first reducer, and the second driving module 30b may include a second actuator and a second reducer. An output end of the first actuator may be connected, directly or indirectly, to an input end of the first reducer, and an output end of the second actuator may be connected, directly or indirectly, to an input end of the second reducer.

According to an embodiment, a processor (e.g., the processor 310 described below) may generate a first control signal (e.g., a first control signal τₗ(t) described below) to control the first driving module 30a, and control the first driving module 30a through the first control signal to generate an external force (or torque) corresponding to the first control signal. The first driving module 30a may generate a torque by driving the first actuator according to the first control signal. The torque generated through the first actuator may be reduced by the first reducer. The torque reduced by the first reducer may rotate the first leg driving frame 70a. The torque reduced by the first reducer may be provided, for example, to the left leg of the user through the first leg driving frame 70a.

According to an embodiment, the processor (e.g., the processor 310 described below) may generate a second control signal (e.g., a second control signal τ_{R}(t) described below) to control the second driving module 30b, and control the second driving module 30b through the second control signal to generate an external force (or torque) corresponding to the second control signal. The second driving module 30b may generate a torque by driving the second actuator according to the second control signal. The torque generated through the second actuator may be reduced by the second reducer. The torque reduced by the second reducer may rotate the second leg driving frame 70b. The torque reduced by the second reducer may be provided, for example, to the right leg of the user through the second leg driving frame 70b.

Each “processor” herein includes processing circuitry, and/or may include multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

According to an embodiment, the leg driving frames 70a and 70b may support the legs (e.g., thighs) of the user when the wearable device 200 is worn on the legs of the user. The leg driving frames 70a and 70b may include the first leg driving frame 70a configured to support the left leg of the user and the second leg driving frame 70b configured to support the right leg of the user.

According to an embodiment, the leg driving frames 70a and 70b may, for example, transmit the torques generated by the driving modules 30a and 30b (e.g., the torques reduced by the reducers) to the thighs of the user. As one end portions of the leg driving frames 70a and 70b are connected, directly or indirectly, to the driving modules 30a and 30b to rotate, and the other end portions of the leg driving frames 70a and 70b are connected, directly or indirectly, to the thigh fastening portions 40a and 40b, the leg driving frames 70a and 70b may transmit the torques generated by the driving modules 30a and 30b to the thighs of the user while supporting the thighs of the user. For example, the leg driving frames 70a and 70b may push or pull the thighs of the user. The leg driving frames 70a and 70b may extend in the longitudinal direction of the thighs of the user. The leg driving frames 70a and 70b may be bent to surround at least a portion of the circumferences of the thighs of the user.

According to an embodiment, the thigh fastening portions 40a and 40b may be connected, directly or indirectly, to the leg driving frames 70a and 70b and may fasten the leg driving frames 70a and 70b to the thighs. The thigh fastening portions 40a and 40b may include a first thigh fastening portion 40a configured to fasten the first leg driving frame 70a to the left thigh of the user and a second thigh fastening portion 40b configured to fasten the second leg driving frame 70b to the right thigh of the user.

According to an embodiment, the first thigh fastening portion 40a may include a first cover, a first fastening frame, and a first strap, and the second thigh fastening portion 40b may include a second cover, a second fastening frame, and a second strap. The first cover and the second cover may be arranged on one sides of the thighs of the user. The first cover and the second cover may be arranged on the front surfaces of the thighs of the user. The first cover and the second cover may be arranged in the circumferential directions of the thighs of the user. The first cover and the second cover may extend to both sides from the other end portions of the leg driving frames 70a and 70b and may include curved surfaces corresponding to the thighs of the user. One ends of the first cover and the second cover may be connected, directly or indirectly, to the fastening frames, and the other ends thereof may be connected, directly or indirectly, to the straps.

According to an embodiment, the first fastening frame and the second fastening frame may be arranged, for example, to surround at least some portions of the circumferences of the thighs of the user, thereby preventing or reducing chances of the thighs of the user from being separated from the leg driving frames 70a and 70b. The first fastening frame may have a fastening structure that connects the first cover and the first strap, and the second fastening frame may have a fastening structure that connects the second cover and the second strap.

According to an embodiment, the first strap may enclose the remaining portion of the circumference of the left thigh of the user that is not covered by the first cover and the first fastening frame, and the second strap may enclose the remaining portion of the circumference of the right thigh of the user that is not covered by the second cover and the second fastening frame. The first strap and the second strap may include, for example, an elastic material (e.g., a band).

According to an embodiment, the main belt 50 may be connected, directly or indirectly, to the base frame 20. The main belt 50 may include a first main belt 50a configured to enclose the left abdomen of the user while the user is wearing the wearable device 200, and a second main belt 50b configured to enclose the right abdomen of the user while the user is wearing the wearable device 200. The first main belt 50a may be formed in a shape having a longer length than the second main belt 50b, but is not limited thereto, and the first main belt 50a may be formed in a shape having the same length as or a shorter length than the second main belt 50b. The first main belt 50a and the second main belt 50b may be connected, directly or indirectly, to both end portions of the base frame 20, respectively. The main belt 50 may be bent in a direction to surround the abdomen of the user when the body of the user is inserted in such a direction that it is accommodated in the wearable device 200. The first main belt 50a and the second main belt 50b may be connected, directly or indirectly, to each other while the user is wearing the wearable device 200. The main belt 50 may distribute a portion of the weight of the wearable device 200 to the abdomen of the user while the user is wearing the wearable device 200.

Referring to FIG. 2B, the base body 10 may be mounted on the back of the lumbar region of the user and be hung on the hip region of the user, thereby supporting a portion of the weight of the wearable device 200. The first driving module 30a may be arranged on the left lumbar region of the user. The base frame 20 may extend from the end portion of the base body 10 and be inclined in a direction toward the first driving module 30a. The first main belt 50a mounted on the base frame 20 may surround the left abdomen of the user.

FIGS. 3A and 3B are block diagrams illustrating examples of a configuration of a wearable device according to an embodiment.

According to an embodiment, a wearable device 300 of FIG. 3A may include the processor 310, angle sensors 320 and 320-1, a battery 330, a power management integrated circuit (PMIC) 340, a memory 350, an IMU 360, motor driver circuits 370 and 370-1, motors 380 and 380-1 (e.g., the first and second actuators described with reference to FIG. 2A), and a communication module 390.

Although the plurality of angle sensors 320 and 320-1, the plurality of motor driver circuits 370 and 370-1, and the plurality of motors 380 and 380-1 are shown in FIG. 3, which is merely an example, the wearable device 300-1 in the example shown in FIG. 3B may include a single angle sensor 320, a single motor driver circuit 370, and a single motor 380. Also, according to the implementation, the wearable devices 300 and 300-1 may include a plurality of processors. The number of motor driver circuits, the number of motors, or the number of processors may vary depending on a body part on which the wearable devices 300 and 300-1 are worn.

The wearable device 300 in FIG. 3A and the wearable device 300-1 of FIG. 3B may be examples of the wearable device 110 and the wearable device 200.

According to an embodiment, the angle sensor 320, the motor driver circuit 370, and the motor 380 may be included in the first driving module 30a of FIG. 2A, and the angle sensor 320-1, the motor driver circuit 370-1, and the motor 380-1 may be included in the second driving module 30b of FIG. 2A.

According to an embodiment, the angle sensor 320 and the angle sensor 320-1 may each correspond to an encoder or a Hall sensor, but are not limited thereto.

According to an embodiment, the angle sensor 320 may measure (or sense) at least one of the angle, angular velocity, or angular acceleration of a first joint (e.g., the left hip joint, the left knee joint, etc.) of the user. The angle sensor 320 may transmit first angle data (or first angle values) (e.g., qₗ(t)) generated (or obtained) by measuring the angle of the first joint to the processor 310. For example, the angle sensor 320 may measure (or obtain) the first angle data (or the first angle values) by measuring the left hip joint angle of the user, and transmit the first angle data (or the first angle values) to the processor 310.

According to an embodiment, the angle sensor 320-1 may measure (or sense) at least one of the angle, angular velocity, or angular acceleration of a second joint (e.g., the right hip joint, the right knee joint, etc.) of the user. The angle sensor 320-1 may transmit second angle data (or second angle values) (e.g., qᵣ(t)) generated (or obtained) by measuring the angle of the second joint to the processor 310. For example, the angle sensor 320-1 may measure (or obtain) the second angle data (or the second angle values) by measuring the right hip joint angle of the user, and transmit the second angle data (or the second angle values) to the processor 310.

According to an embodiment, the angle sensor 320 and the angle sensor 320-1 may additionally measure the knee angles and ankle angles of the user according to the positions of the angle sensor 320 and the angle sensor 320-1.

According to an embodiment, the wearable devices 300 and 300-1 may include a potentiometer. The potentiometer may sense an R-axis joint angle, an L-axis joint angle, an R-axis joint angular velocity, and an L-axis joint angular velocity according to a walking motion of the user. In this example, the R and L axes may be reference axes for the right leg and the left leg of the user, respectively. For example, the R/L axis may be set to be vertical to the ground and set such that a front side of a body of a person has a negative value and a rear side of the body has a positive value.

According to an embodiment, the PMIC 340 may charge the battery 330 using power supplied from an external power source. For example, the external power source and the wearable devices 300 and 300-1 may be connected through a cable (e.g., a universal serial bus (USB) cable, etc.). The PMIC 340 may receive power from the external power source through the cable, and charge the battery 330 using the received power. According to embodiments, the PMIC 340 may charge the battery 330 through a wireless charging method. According to an embodiment, the PMIC 340 may transmit the power stored in the battery 330 to the components (e.g., the processor 310, the angle sensors 320 and 320-1, the memory 350, the IMU 360, the motors 380 and 380-1, etc.) in the wearable devices 300 and 300-1. The PMIC 340 may, for example, adjust the power stored in the battery 330 to a voltage or current level suitable for the components in the wearable device 300. The PMIC 340 may include, for example, a converter (e.g., a direct current (DC)-DC converter) or a regulator (e.g., a low-dropout (LDO) regulator or a switching regulator) configured to perform the adjustment described above.

According to an embodiment, the PMIC 340 may determine state information (e.g., a state of charge, a state of health, an overvoltage, a low voltage, an overcurrent, an overcharge, an overdischarge, an overheating, a short circuit, or a swelling) of the battery 330, and transmit the state information of the battery 330 to the processor 310. The processor 310 may control to provide the state information of the battery 330 to the user. For example, the processor 310 may output the state information of the battery 330 through at least one of a sound output module (e.g., a speaker), a vibration output module, or a display module (e.g., the lighting unit 60). For example, the processor 310 may transmit the state information of the battery 330 to the electronic device 120 through the communication module 390, and the electronic device 120 may display the state information of the battery 330 on the display.

According to an embodiment, the IMU 360 may obtain or measure the acceleration and/or rotation angle of the user. For example, the IMU 360 may measure or obtain 3-axis (e.g., x-axis, y-axis, and z-axis) accelerations and/or rotation angles (e.g., roll, pitch, and yaw) according to a motion (e.g., walking or exercise) of the user. The IMU 360 may transmit the obtained acceleration and/or rotation angle to the processor 310.

According to an embodiment, the processor 310 may control the overall operation of the wearable devices 300 and 300-1.

According to an embodiment, the processor 310 may, for example, control the components (e.g., the motor driver circuits 370 and 370-1, etc.) in the wearable devices 300 and 300-1 by executing software (e.g., a program or instructions) stored in the memory 350, and perform various data processing or computation. As at least a portion of the data processing or computation, the processor 310 may store data received from other components (e.g., the IMU 360, the angle sensors 320 and 320-1, etc.) in the memory 350, and process the instructions or data stored in the memory 350.

According to an embodiment, the processor 310 may control the driving module 30 (e.g., the motor driver circuits 370 and 370-1) so that the driving module 30 (e.g., the motors 380 and 380-1) may generate a torque. As described in detail below, the processor 310 may generate a filter input (or filter input data) based on the difference between the first angle data and the second angle data. This filter input may also be referred to as first data. The processor 310 may generate a filter output (or filter output data) having a phase delayed from the phase of the filter input by performing filtering (e.g., filtering through a low-pass filter) on the filter input. This filter output may also be referred to as second data. The processor 310 may delay the phase of the filter input through a filter so that a change timepoint of a torque rotation direction of the driving module 30 may correspond to a peak timepoint of the filter input. The processor 310 may control the driving module 30 based on the generated filter output so that a torque based on the generated filter output may be generated by the driving module 30. The driving module 30 (e.g., the motor driver circuits 370 and 370-1) may drive the motors 380 and 380-1 under the control of the processor 310 to generate a torque.

According to an embodiment, the communication module 390 may support the establishment of a direct (or wired) communication channel or a wireless communication channel between the wearable device 300, 300-1 and an external electronic device, and support the communication through the established communication channel. The communication module 390 may include one or more communication processors configured to support direct (or wired) communication or wireless communication. According to an embodiment, the communication module 390 may include a wireless communication module (e.g., a cellular communication module, a short-range wireless communication module, or a global navigation satellite system (GNSS) communication module) or a wired communication module (e.g., a local area network (LAN) communication module or a power line communication (PLC) module). A corresponding one of these communication modules may communicate with the external electronic device via a first network (e.g., a short-range communication network such as Bluetooth™, wireless-fidelity (Wi-Fi) direct, or infrared data association (IrDA)) or a second network (e.g., a legacy cellular network, a 5G network, a next-generation communication network, the Internet, or a computer network). These various types of communication modules may be implemented as a single component (e.g., a single chip), or may be implemented as multiple components (e.g., multiple chips) separate from each other.

According to an embodiment, the wearable devices 300 and 300-1 may include a display module. The display module may include, for example, a display and/or a lighting unit (e.g., the lighting unit 60 of FIG. 2A). The processor 310 may control the display module so that the display module may provide visual feedback to the user.

According to an embodiment, the wearable devices 300 and 300-1 may include a sound output module. The sound output module may include, for example, a speaker. The processor 310 may control the sound output module so that the sound output module may provide auditory feedback to the user.

According to an embodiment, the wearable devices 300 and 300-1 may include a vibration output module. The vibration output module may include, for example, a vibration motor. The processor 310 may control the vibration output module so that the vibration output module may provide tactile feedback (or haptic feedback) to the user.

According to an embodiment, at least one of the processor 310, the battery 330, the PMIC 340, the memory 350, the IMU 360, the communication module 390, the display module, the sound output module, or the vibration output module, or a combination thereof may be positioned in the base body 10 of FIGS. 2A and 2B.

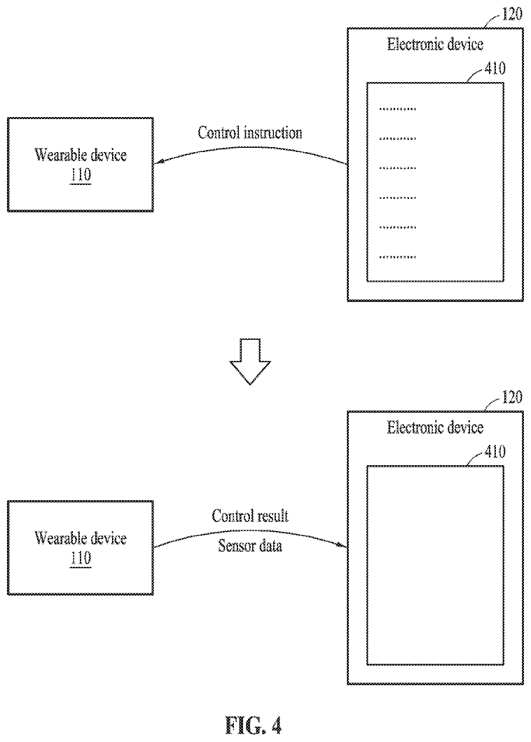

FIG. 4 is a diagram illustrating an interaction between a wearable device and an electronic device according to an embodiment.

Referring to FIG. 4, the wearable device 110 may communicate with the electronic device 120 (e.g., a smart phone or smart watch). For example, the electronic device 120 may be a user terminal of the user who uses the wearable device 110 or a controller device dedicated to the wearable device 110. According to an embodiment, the wearable device 110 and the electronic device 120 may be connected to each other through short-range wireless communication (e.g., Bluetooth™ or Wi-Fi communication).

According to an embodiment, the electronic device 120 may verify a state of the wearable device 110 or execute an application to control or operate the wearable device 110. A screen of a user interface (UI) may be displayed to control an operation of the wearable device 110 on a display 410 of the electronic device 120 through the execution of the application. The UI may be, for example, a graphical user interface (GUI).

According to an embodiment, the wearable device 110 may receive control information indicating a level value of an exercise intensity (e.g., an exercise intensity of the target exercise) of the user and/or an operation mode (e.g., an assistance mode or a resistance mode) of the wearable device 110 from the electronic device 120 and/or the other wearable device 130.

For example, the level value of the exercise intensity may be a value related to the magnitude of an external force (or a torque) provided by the wearable device 110 to the user. The higher the level value, the stronger the external force (or torque) may be provided to the user. The wearable device 110 may determine the size of a gain, to be described later, using the level value. For example, the wearable device 110 may store a table in which level values and the sizes of gains are mapped. Table 1 below shows an example of the table.

TABLE 1

| Exercise intensity | Size of gain |
|---|---|
| Level value = 1 | K₁ |
| Level value = 2 | K₂ |
| Level value = 3 | K₃ |
| Level value = 4 | K₄ |
| Level value = 5 | K₅ |

In Table 1 above, K₁ to K₅ may be greater than “0”.

In Table 1 above, the exercise intensity is classified into five levels, but this is only an example. The exercise intensity is not limited to the five levels.

The assistance mode may be an operation mode in which the wearable device 110 provides an assistance force (or assistance torque) to the user. The wearable device 110 may determine the operation mode to be the assistance mode according to the received control information. The wearable device 110 may determine the sign of the gain to be a first sign (e.g., plus) in the assistance mode. The resistance mode may be an operation mode in which the wearable device 110 provides a resistance force (or resistance torque) to the user. The wearable device 110 may determine the operation mode to be the resistance mode according to the received control information. The wearable device 110 may determine the sign of the gain to be a second sign (e.g., minus) in the resistance mode.

For example, the wearable device 110 may receive the control information (e.g., level value=3/operation mode=assistance mode) from the electronic device 120 and/or the other wearable device 130. The wearable device 110 may determine the value of the gain to be a positive value (e.g., +K₃) based on the received control information (e.g., level value=3/operation mode=assistance mode). As another example, the wearable device 110 may receive the control information (e.g., level value=2/operation mode=resistance mode) from the electronic device 120 and/or the other wearable device 130. The wearable device 110 may determine the value of the gain to be a negative value (e.g., −K₂) based on the received control information (e.g., level value=2/operation mode=resistance mode).

According to an embodiment, the wearable device 110 may transmit sensor data measured by a sensor (e.g., at least one of the angle sensor 320, the angle sensor 320-1, or the IMU 360) of the wearable device 110 to the electronic device 120. The electronic device 120 may provide the user with result information (e.g., walking ability information, exercise ability information, or exercise posture evaluation information) derived by analyzing the sensor data through the GUI screen.

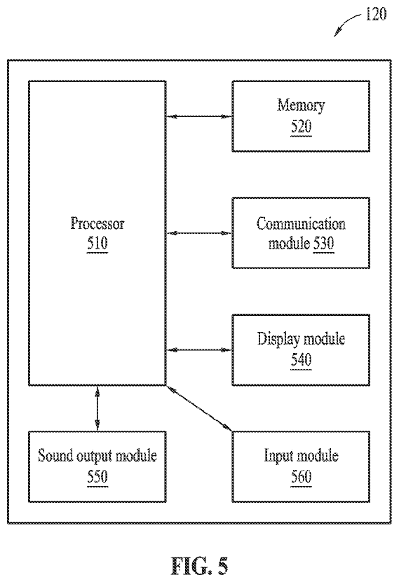

FIG. 5 is a diagram illustrating an example of a configuration of an electronic device according to an embodiment.

Referring to FIG. 5, the electronic device 120 may include a processor 510, a memory 520, a communication module 530, a display module 540, a sound output module 550, and an input module 560. In an embodiment, at least one (e.g., the sound output module 550) of these components may be omitted from the electronic device 120, or one or more other components (e.g., a sensor module, a haptic module, and a battery) may be added thereto.

The processor 510 may control at least one other component (e.g., a hardware or software component) of the electronic device 120, and may perform a variety of data processing or computation. According to an embodiment, as at least part of data processing or computation, the processor 510 may store instructions or data received from another component (e.g., the communication module 530) in the memory 520, process the instructions or data stored in the memory 520, and store result data obtained as a result of processing in the memory 520.

According to an embodiment, the processor 510 may include a main processor (e.g., a central processing unit (CPU) or an application processor (AP)) or an auxiliary processor (e.g., a graphics processing unit (GPU)), a neural processing unit (NPU), an image signal processor (ISP), a sensor hub processor, or a communication processor (CP)) that is operable independently of, or in conjunction with the main processor.

The memory 520 may store a variety of data used by at least one component (e.g., the processor 510 or the communication module 530) of the electronic device 120. The data may include, for example, a program (e.g., an application), and input data or output data for instructions related thereto. The memory 520 may include at least one instruction executable by the processor 510. The memory 520 may include a volatile memory or a non-volatile memory.

The communication module 530 may support the establishment of a direct (e.g., wired) communication channel or a wireless communication channel between the electronic device 120 and another electronic device (e.g., the wearable device 110, the other wearable device 130, or the server 140), and support the communication through the established communication channel. The communication module 530 may include a communication circuit configured to perform a communication function. The communication module 530 may include one or more communication processors that are operable independently from the processor 510 (e.g., the application processor) and support direct (e.g., wired) communication or wireless communication. According to an embodiment, the communication module 530 may include a wireless communication module configured to perform wireless communication (e.g., a Bluetooth communication module, a cellular communication module, a Wi-Fi communication module, or a GNSS communication module) or a wired communication module (e.g., a LAN communication module or a power line communication (PLC) module). For example, the communication module 530 may transmit a control instruction to the wearable device 110 and receive, from the wearable device 110, at least one of sensor data including body motion information of the user who is wearing the wearable device 110, state data of the wearable device 110, or control result data corresponding to the control instruction.

The display module 540 may visually provide information to the outside (e.g., the user) of the electronic device 120. The display module 540 may include, for example, a liquid-crystal display (LCD) or organic light-emitting diode (OLED) display, a hologram device, or a projector device. The display module 540 may further include a control circuit configured to control the driving of a display. In an embodiment, the display module 540 may further include a touch sensor set to sense a touch or a pressure sensor set to sense the intensity of a force generated by the touch. The display module 540 may output a user interface screen for controlling the wearable device 110 or providing a variety of information (e.g., exercise evaluation information and setting information of the wearable device 110).

The sound output module 550 may output sound signals to the outside of the electronic device 120. The sound output module 550 may include a speaker configured to play back a guiding sound signal (e.g., an operation start sound or an operation error alarm), music content, or a guiding voice based on the state of the wearable device 110. For example, when it is determined that the wearable device 110 is not normally worn on the body of the user, the sound output module 550 may output a guiding voice to inform the user is wearing the wearable device 110 abnormally or to guide the user to wear the wearable device 110 normally.

The input module 560 may receive a command or data to be used by a component (e.g., the processor 510) of the electronic device 120 from the outside (e.g., the user) of the electronic device 120. The input module 560 may include an input component circuit and may receive a user input. The input module 560 may include, for example, a touch recognition circuit for recognizing a touch on a key (e.g., a button) and/or a screen.

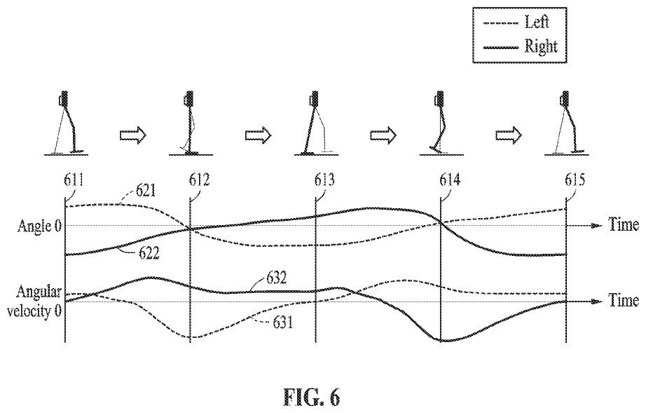

FIG. 6 is a diagram illustrating an example of motion information of a user obtained by a wearable device according to an embodiment.

In the example shown in FIG. 6, a user may perform walking, and the wearable device 110 may detect a first joint angle (e.g., the left hip joint angle) and a second joint angle (e.g., the right hip joint angle) of the walking user. An example of first angle data (or first angle values) qₗ(t) 621 obtained by the wearable device 110 by measuring the first joint angle of the user and an example of second angle data (or second angle values) qᵣ(t) 622 obtained by the wearable device 110 by measuring the second joint angle of the user are shown in FIG. 6. The first joint angle and the second joint angle may change depending on a motion of the user. The first angle data (or first angle values) 621 may correspond to, for example, time series data (or time series angle values) indicating changes in the first joint angle, and the second angle data 622 may correspond to, for example, time series data (or time series angle values) indicating changes in the second joint angle.

An example of first angular velocity data 631 obtained by the wearable device 110 by calculating the angular velocity of the first joint angle based on the first angle data 621 and an example of second angular velocity data 632 obtained by the wearable device 110 by calculating the angular velocity of the second joint angle based on the second angle data 622 are shown in FIG. 6. The first angular velocity data 631 may correspond to, for example, time series data (or time series angular velocity values) indicating changes in the angular velocity of the first joint, and the second angular velocity data 632 may correspond to, for example, time series data (or time series angular velocity values) indicating changes in the angular velocity of the second joint.

The motion information of the user obtained by the wearable device 110 may include, for example, at least one of the first angle data 621, the second angle data 622, the first angular velocity data 631, or the second angular velocity data 632.

At timepoint 611, the second joint (e.g., the right hip joint) of the user may be in a state of being fully extended forward. In the example shown in FIG. 6, the front from the user may be an area in which the angle value is negative, and the rear from the user may be an area in which the angle value is positive. Since the second joint (e.g., the right hip joint) of the user is in a state of being fully extended forward at timepoint 611, the second angle data 622 at timepoint 611 may have a negative value (e.g., a negative peak value). Additionally, the angular velocity value of the second angular velocity data 632 at timepoint 611 may be “0”. The angular velocity value of the second angular velocity data 632 at timepoint 611 may be “0”, and the angular velocity value of the second angular velocity data 632 after timepoint 611 may be positive. Thus, the processor 310 may recognize through the second angular velocity data 632 (or the angular velocity values of the second angular velocity data 632) that the rotation direction of the second joint changes from a first direction (e.g., the counterclockwise direction of FIG. 6) to a second direction (e.g., the clockwise direction of FIG. 6).

Between timepoint 611 and timepoint 612, there may be a state in which the first joint (e.g., the left hip joint) of the user is fully extended backward (e.g., a state in which the angular velocity value of the first angular velocity data 631 is “0”). The first joint of the user may rotate forward after the state in which the first joint of the user is fully extended backward. The processor 310 may recognize, through the first angular velocity data 631 (or the angular velocity values of the first angular velocity data 631) between timepoint 611 and timepoint 612, that the rotation direction of the first joint of the user changes from the second direction (e.g., the clockwise direction of FIG. 6) to the first direction (e.g., the counterclockwise direction of FIG. 6) after the state in which the first joint of the user is fully extended backward.

At timepoint 612, the first joint (e.g., the left hip joint) of the user may rotate forward, and the left leg and the right leg of the user may cross. At timepoint 612, the left leg that is rotating forward and the right leg may cross. At timepoint 612, the angle value of the first angle data 621 and the angle value of the second angle data 622 may be the same. The processor 310 may recognize the timepoint when the difference between the angle value of the first angle data 621 and the angle value of the second angle data 622 is “O” as the timepoint when both legs of the user cross.

At timepoint 613, the first joint of the user may be in a state of being fully extended forward. At timepoint 613, the first angle data 621 may have a peak value (e.g., a negative peak value), and the angular velocity value of the first angular velocity data 631 may be “0”. The processor 310 may recognize, through the first angular velocity data 631 (or the angular velocity values of the first angular velocity data 631), that the rotation direction of the first joint of the user changes from the first direction (e.g., the counterclockwise direction of FIG. 6) to the second direction (e.g., the clockwise direction of FIG. 6) after timepoint 613.

Between timepoint 613 and timepoint 614, there may be a state in which the second joint of the user is fully extended backward (e.g., a state in which the angular velocity value of the second angular velocity data 632 is “0”). The second joint of the user may rotate forward after the state in which the second joint of the user is fully extended backward. The processor 310 may recognize, through the second angular velocity data 632 (or the angular velocity values of the second angular velocity data 632) between timepoint 613 and timepoint 614, that the rotation direction of the second joint of the user changes from the second direction (e.g., the clockwise direction of FIG. 6) to the first direction (e.g., the counterclockwise direction of FIG. 6) after the state in which the second joint of the user is fully extended backward.

At timepoint 614, the second joint of the user may rotate forward, and the right leg and the left leg of the user may cross. At timepoint 614, the right leg that is rotating forward and the left leg may cross. At timepoint 614, the angle value of the first angle data 621 and the angle value of the second angle data 622 may be the same. The processor 310 may recognize the timepoint when the difference between the angle value of the first angle data 621 and the angle value of the second angle data 622 is “O” as the timepoint when both legs of the user cross.

At timepoint 615, the second joint of the user may be in a state of being fully extended forward. At timepoint 615, the second angle data 622 may have a peak value (e.g., a negative peak value), and the angular velocity value of the second angular velocity data 632 may be “0”.

According to an embodiment, the wearable device 110 may determine a walking period (or one period of a walking cycle) indicating a period in which the walking postures of the user are repeated, using at least a portion of the motion information (e.g., the first angle data 621, the second angle data 622, the first angular velocity data 631, and/or the second angular velocity data 632) of the user. For example, the user may perform a walking posture of fully rotating the right leg forward at timepoint 611 of FIG. 6, and the user may perform the walking posture of fully rotating the right leg forward again at timepoint 615. The walking posture at timepoint 611 may be repeated at timepoint 615. The processor 310 may recognize each of timepoint 611 and timepoint 615 through at least one of the second angle data 622 or the second angular velocity data 632, and determine a time interval between timepoint 611 and timepoint 615 to be the walking period of the user. The embodiment of determining the walking period of the user is not limited to the example described above.

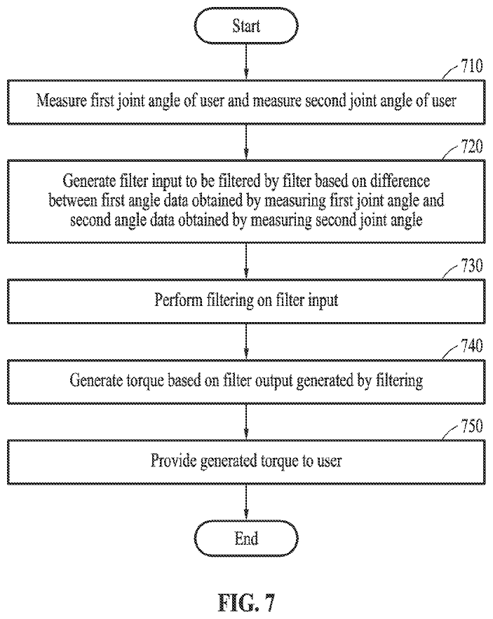

FIG. 7 is a flowchart illustrating an example of an operating method of a wearable device according to an embodiment.

Referring to FIG. 7, in operation 710, the wearable device 110 may measure a first joint angle of a user and measure a second joint angle of the user. For example, the angle sensor 320 may measure the first joint angle, and transmit first angle data (e.g., the first angle data 621 of FIG. 6) obtained by measuring the first joint angle to the processor 310. The angle sensor 320-1 may measure the second joint angle, and transmit second angle data (e.g., the second angle data 622 of FIG. 6) obtained by measuring the second joint angle to the processor 310.

In operation 720, the wearable device 110 may generate a filter input (or filter input data or filter input values) to be filtered by a filter based on the difference between the first angle data qₗ(t) obtained by measuring the first joint angle and second angle data qᵣ(t) obtained by measuring the second joint angle.

According to an embodiment, the processor 310 may generate the filter input using the difference between the result (e.g., sin qₗ(t)) of applying a trigonometric function (e.g., sine (sin) function) to the second angle data qᵣ(t) and the result (e.g., sin qₗ(t)) of applying a trigonometric function (e.g., sin function) to the first angle data q (t). For example, the processor 310 may generate the filter input y₁ (t) corresponding to the difference between sin qᵣ(t) and sin qₗ(t) according to the following Equation 1.

$${y}_{1} ( t ) = sin {q}_{r} ( t ) - sin {q}_{l} ( t ) \qquad [ Equation 1 ]$$

According to an embodiment, the processor 310 may generate the filter input using the difference between the result (e.g., tan h qᵣ(t)) of applying a hyperbolic function (e.g., hyperbolic tangent (tan h) function) to the second angle data qᵣ(t) and the result (e.g., tan h qₗ(t)) of applying a hyperbolic function (e.g., tan h function) to the first angle data q (t). For example, the processor 310 may generate the filter input y₂ (t) corresponding to the difference between tan h qᵣ(t) and tan h qₗ(t) according to the following Equation 2.

$${y}_{2} ( t ) = tan h {q}_{r} ( t ) - tan h {q}_{l} ( t ) \qquad [ Equation 2 ]$$

According to an embodiment, the processor 310 may generate the filter input by dividing the difference qᵣ(t)-qₗ(t) between the second angle data qᵣ(t) and the first angle data qₗ(t) by R. Here, R may denote the maximum and/or a high angle at which the hip joint can rotate. For example, the processor 310 may generate the filter input y₃(t) according to the following Equation 3.

$${y}_{3} ( t ) = \frac{( {q}_{r} ( t ) - {q}_{l} ( t ) )}{R} \qquad [ Equation 3 ]$$

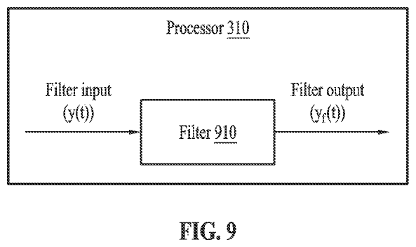

In operation 730, the wearable device 110 may perform filtering on the filter input y(t) (e.g., y₁(t), y₂(t), or y₃(t)). For example, the processor 310 may perform filtering on the filter input y(t) through a filter (e.g., a low-pass filter). As described in detail below, the filter (e.g. the low-pass filter) may have a characteristic of delaying the phase of a given input. A filter output y_{f}(t) generated by filtering may have a phase delayed by a first phase value (e.g., 90 degrees) from the phase of the filter input y(t) by the filter. The processor 310 may delay the phase of the filter input by the first phase value through the filter, so that a change timepoint of the torque rotation direction of the driving module 30 (e.g., a timepoint of change from the first direction to the second direction or a timepoint of change from the second direction to the first direction) may correspond to a change timepoint of the rotation direction of a joint. Filtering in operation 730 will be described later with reference to FIGS. 9, 10, and 11.

In operation 740, the wearable device 110 may generate a torque (e.g., an assistance torque or a resistance torque) based on the filter output generated by filtering.

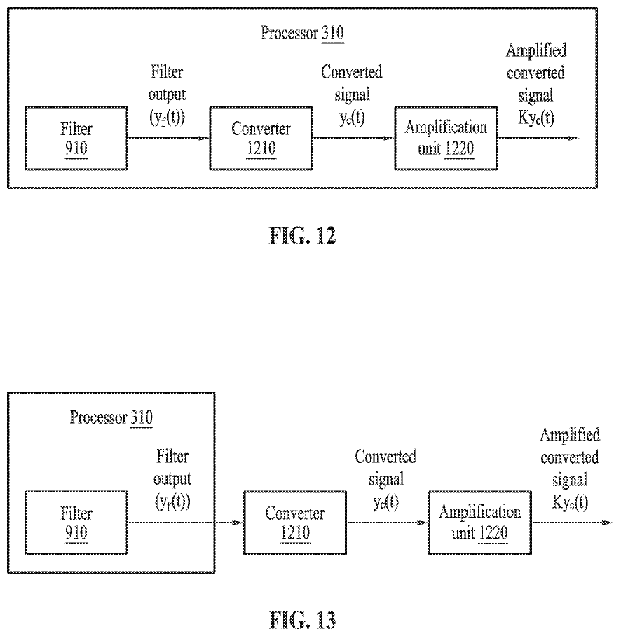

According to an embodiment, the filter output may be, for example, a digital signal. In operation 740, the processor 310 may generate a converted signal (e.g., y(t) described below) by converting the filter output through a converter (e.g., a converter 1210 described with reference to FIGS. 12 and 13). The generated converted signal may be, for example, an analog signal (e.g. a voltage signal or a current signal). The processor 310 may amplify the converted signal through an amplification unit (e.g., an amplification unit 1220 described with reference to FIGS. 12 and 13), transmit the amplified converted signal to the driving module 30, and control the driving module 30 so that a torque corresponding to the amplified converted signal (e.g., Ky_{c}(t) described below) may be generated.

In operation 750, the wearable device 110 may provide the generated torque to the user. Changing the rotation direction of the torque at the same time as the timepoint when the rotation direction of the joint of the user changes may allow the user to feel a natural torque suitable for the motion of the user. According to an embodiment, the wearable device 110 may change the rotation direction of the torque at the timepoint when the rotation direction of the joint of the user changes through phase delay so that a natural torque suitable for the motion of the user may be provided to the user. The wearable device 110 may generate a filter output having a phase delayed by the first phase value from the phase of the filter input through the filter, and generate a torque corresponding to the filter output. Through this phase delay, the wearable device 110 may change the rotation direction of the torque when the rotation direction of the joint of the user changes.

According to an embodiment, the wearable device 110 may operate in an assistance mode. In the assistance mode, the wearable device 110 may provide a torque in the rotation direction the same as the rotation direction of the joint to the user. The wearable device 110 may change the rotation direction of the torque from the first direction to the second direction at the timepoint when the rotation direction of the joint of the user changes from the first direction to the second direction. The wearable device 110 may operate in a resistance mode. In the resistance mode, the wearable device 110 may provide a torque in the rotation direction opposite to the rotation direction of the joint to the user. The wearable device 110 may change the rotation direction of the torque from the second direction to the first direction at the timepoint when the rotation direction of the joint of the user changes from the first direction to the second direction.

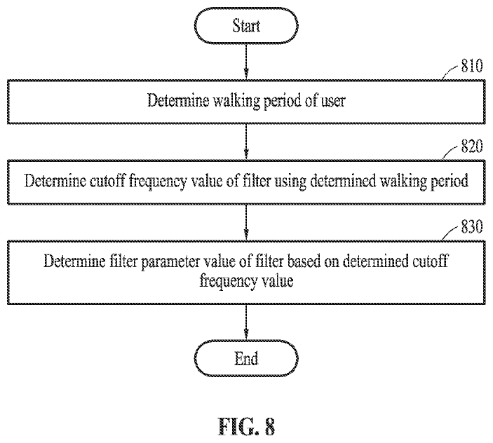

FIG. 8 is a flowchart illustrating an example of an operating method of a wearable device according to an embodiment.

Referring to FIG. 8, in operation 810, the wearable device 110 may determine a walking period of the user. The wearable device 110 may determine the walking period of the user using the motion information of the user.

For example, as described with reference to FIG. 6, in operation 810, the processor 310 may recognize each of timepoint 611 and timepoint 615 through at least one of the second angle data 622 or the second angular velocity data 632, and determine the time interval between timepoint 611 and timepoint 615 to be the walking period of the user.

As another example, in operation 810, the processor 310 may determine the time interval between two adjacent positive peak values of the first angle data 621, the time interval between two adjacent positive peak values of the second angle data 622, the time interval between two adjacent positive peak values of the first angular velocity data 631, or the time interval between two adjacent positive peak values of the second angular velocity data 632 to be the walking period of the user.

As another example, in operation 810, the processor 310 may recognize the cross timepoint (e.g., timepoint 612 of FIG. 6) of a first leg (e.g., the left leg) that is rotating forward and a second leg (e.g., the right leg) using the first angle data 621 and the second angle data 622, and recognize the cross timepoint at which the first leg that is rotating forward and the second leg cross again using the first angle data 621 and the second angle data 622. The processor 310 may determine the time interval between the respective recognized cross timepoints to be the walking period of the user.

As another example, in operation 810, the processor 310 may receive information (e.g., three-axis acceleration and/or rotation angle) obtained by the IMU 360 from the IMU 360. The processor 310 may recognize the timepoint when one foot of the user touches the ground and the timepoint when the one foot touches the ground again using at least a portion of the information received from the IMU 360, and determine the time interval between the respective recognized timepoints to be the walking period of the user.

In operation 820, the wearable device 110 may determine a cutoff frequency value of the filter using the determined walking period.

For example, the processor 310 may determine the cutoff frequency value ω_{c} of the filter according to an equation

$$ω = \frac{2 π}{T} [ rad / s ] .$$

Here, ω may denote the angular frequency, and T may denote the walking period of the user. The reciprocal of the walking period T of the user may be a walking frequency value of the user. The filter may have the walking frequency value of the user as the cutoff frequency value.

In operation 830, the wearable device 110 may determine a filter parameter value of the filter based on the determined cutoff frequency value.

The filter output of a filter (e.g., a second-order Butterworth low-pass filter) may be expressed, for example, as Equation 4 below.

$$[ Equation 4 ]$$

$${y}_{f} ( t ) = - {a}_{1} {y}_{f} ( t - 1 ) - {a}_{2} {y}_{f} ( t - 2 ) + {b}_{0} y ( t ) + {b}_{1} y ( t - 1 ) + {b}_{2} y ( t - 2 )$$

In Equation 4 above, a₁, a₂, b₀, b₁, and b₂ may denote filter parameters (or filter coefficients), respectively. a₁ and a₂ may be expressed by Equation 5 below, and b₀, b₁, and b₂ may be expressed as Equation 6 below.

$${a}_{1} = \frac{2 ( 1 - {ω}^{2} )}{1 + \sqrt{2} ω + {ω}^{2}} , \qquad [ Equation 5 ]$$

$${a}_{2} = \frac{- 1 + \sqrt{2} ω - {ω}^{2}}{1 + \sqrt{2} ω + {ω}^{2}}$$

$${b}_{0} = \frac{{ω}^{2}}{1 + \sqrt{2} ω + {ω}^{2}} , \qquad [ Equation 6 ]$$

$${b}_{1} = 2 {b}_{0} ,$$

$${b}_{2} = {b}_{0}$$

The processor 310 may determine the value of a₁ to be

$$\frac{2 ( 1 - {ω}_{c}^{2} )}{1 + \sqrt{2} {ω}_{c} + {ω}_{c}^{2}}$$

and the value of a₂ to be

$$\frac{- 1 + \sqrt{2} {ω}_{c} - {ω}_{c}^{2}}{1 + \sqrt{2} {ω}_{c} + {ω}_{c}^{2}}$$

using Equation 5 above and the determined cutoff frequency value. The processor 310 may determine the value of b₀ to be

$$\frac{{ω}_{c}^{2}}{1 + \sqrt{2} {ω}_{c} + {ω}_{c}^{2}} ,$$

the value of by to be the value of 2b₀, and the value of b₂ to be the value of b₀ according to Equation 6 above.

As described below, the processor 310 may perform filtering on the filter input using the determined filter parameter values (or the filter coefficient values) (e.g., the values of a₁, a₂, b₀, b₁, and b₂).

Hereinafter, a process of deriving y_{f}(t) of Equation 4 above is described briefly.

A transfer function Bₙ(s) of the second-order low-pass filter (e.g., the second-order Butterworth low-pass filter) may be expressed as Equation 7 below.

$${B}_{2} ( s ) = \frac{1}{{s}^{2} + \sqrt{2} s + 1} \qquad [ Equation 7 ]$$

In Equation 7 above, n may denote the order.

A zero-order hold (ZOH) discretization method (e.g., bilinear transformation) may be applied to the above transfer function Bₙ(s), so that an nth-order Butterworth low-pass filter may be used in a time domain (e.g., a discrete time domain). According to the bilinear transformation, s may be substituted with

$$\frac{1}{ω} \frac{z - 1}{z + 1}$$

in Equation 7 above. Here, ω≙

$$tan ( \frac{{ω}_{c} {T}_{s}}{2} )$$

may be satisfied, and Tₛ may denote the sampling time. Equation 8 below may correspond to the result of bilinear transformation of the transfer function Bₙ(s).

$${B}_{n} ( z ) = \frac{1}{{\{ \frac{1}{ω} \frac{1 - {z}^{- 1}}{1 + {z}^{- 1}} \}}^{2} + \frac{\sqrt{2}}{ω} \frac{1 - {z}^{- 1}}{1 + {z}^{- 1}} + 1} \qquad [ Equation 8 ]$$

Equation 8 above may be expressed as Equation 9 below.

$${B}_{n} ( z ) = \frac{( 1 + 2 {z}^{- 1} + {z}^{- 2} ) {ω}^{2}}{{( 1 - {z}^{- 1} )}^{2} + \sqrt{2} ω ( 1 - {z}^{- 1} ) ( 1 + {z}^{- 1} ) + {{ω}^{2} ( 1 + {z}^{- 1} )}^{2}} \qquad [ Equation 9 ]$$

Equation 9 above may be expressed as Equation 10 below.

$${B}_{n} ( z ) = \frac{{y}_{f} ( z )}{y ( z )} = \frac{{b}_{0} + {b}_{1} {z}^{- 1} + {b}_{2} {z}^{- 2}}{1 + {a}_{1} {z}^{- 1} + {a}_{2} {z}^{- 2}} \qquad [ Equation 10 ]$$

z⁻¹ and z⁻² in the z domain may correspond to (t−1) and (t−2) in the time domain, respectively. Converting y_{f}(z) in Equation 10 above into the time domain, it may be expressed as Equation 4 above.

FIGS. 9, 10, and 11 are diagrams illustrating examples of filtering by a wearable device according to an embodiment.

Referring to FIG. 9, the processor 310 may generate a filter output y_{f}(t) by performing filtering on a filter input y(t) through a filter 910.

The operation of filtering the filter input y(t) through the filter by the processor 310 may include an operation of calculating the value of y_{f}(t) at each timepoint according to Equation 4 above.

For example, t=t₁ may be satisfied, and the value of y_{f}(t₁−1) (e.g., the value of the filter output at timepoint (t₁−1)), the value of y_{f}(t₁−2) (e.g., the value of the filter output at timepoint (t₁−2)), the value of y(t₁−1) (e.g., the value of the filter input at timepoint (t₁−1)), and the value of y(t₁−2) (e.g., the value of the filter input at timepoint (t₁−2)) may be stored in the memory (or buffer) of the wearable device 110. The processor 310 may obtain the value of the filter input when t=t₁ (e.g., the value of y(t₁)), and calculate the value of a₁y_{f}(t₁−1), the value of a₂y_{f}(t₁−2), the value of b₀y(t₁), the value of b₁y(t₁−1), and the value of b₂y(t₁−2). The processor 310 may calculate the value of y_{f}(t₁) according to Equation 4 above, and store the value of y_{f}(t₁) in the buffer (or memory).

t=t₁+1 may be satisfied, the value of y_{f}((t₁+1)−1)(=y_{f}(t₁)) (e.g., the value of the filter output at timepoint t₁), the value of y_{f}((t₁+1)−2)(=y_{f}(t₁−1)) (e.g., the value of the filter output at timepoint (t₁−1)), the value of y((t₁+1)−1) (=y(t₁)) (e.g., the value of the filter input at timepoint t₁), and the value of y((t₁+1)−2) (=y(t₁−1)) (e.g., the value of the filter input at timepoint (t₁−1)) may be stored in the memory (or buffer). The processor 310 may obtain the value of the filter input when t=t₁+1 (e.g., the value of y(t₁+1)), and calculate the value of a₁y_{f}(t₁), the value of a₂y_{f}(t₁−1), the value of b₀y(t₁), the value of b₁y(t₁), and the value of b₂y(t₁−1). The processor 310 may calculate the value of y_{f}(t₁+1) according to Equation 4 above, and store the value of y_{f}(t₁+1) in the buffer (or memory). As described above, the filtering may include an operation of calculating the value of y_{f}(t) at each timepoint according to Equation 4 above by the processor 310.

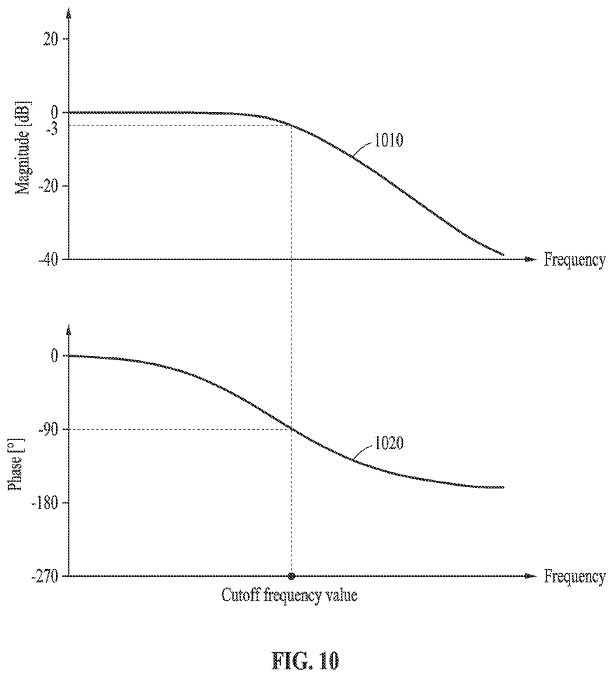

According to an embodiment, the filter 910 may have as walking period (or a walking frequency value) of a user as a cutoff frequency value. The filter 910 may be a second-order low-pass filter (e.g., a second-order Butterworth low-pass filter). The filter 910 may have an amplitude response characteristic that reduces the magnitude of the filter input by 3 dB at the cutoff frequency value (e.g., the walking frequency value of the user) and a phase response characteristic that delays (or shifts) the phase of the filter input by 90 degrees. Examples of an amplitude response characteristic 1010 and a phase response characteristic 1020 of the filter 910 are shown in FIG. 10.

According to an embodiment, as the filter input y(t) is based on data (e.g., the first angle data 621 and the second angle data 622) collected by the walking of the user, the frequency of the filter input y(t) may have the walking frequency value of the user. The filter 910 may have the walking frequency value of the user as the cutoff frequency value. The filter 910 may delay the phase of an input by 90 degrees at the cutoff frequency value according to the phase response characteristic 1020 and thus, may delay the phase of the filter input y(t) having the walking frequency value by 90 degrees, and the filter output y_{f}(t) may have a phase delayed by 90 degrees from the phase of the filter input y(t).

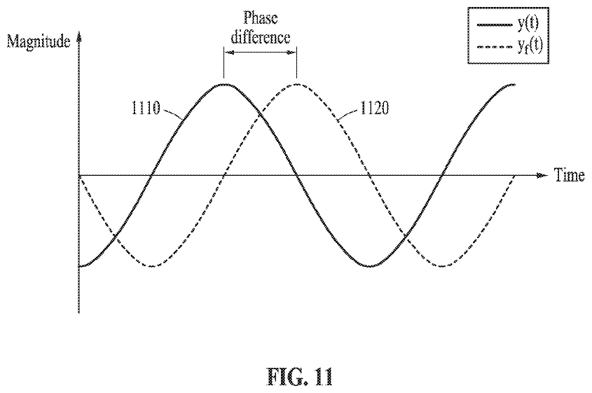

The phase delay may cause a time delay. Expressing the filter input y(t), for example, simply as sin ωt, the filter output y_{f}(t) having the 90-degree delayed phase may be expressed, for example, as sin (ωt+π/2. Plotting the filter input y(t), it may be expressed as a graph 1110 of FIG. 11, and plotting the filter output y_{f}(t), it may be expressed as a graph 1120 of FIG. 11. As in the example shown in FIG. 11, the filter output y_{f}(t) 1120 may be delayed from (or lag behind) the filter input y(t) 1110 by a time value corresponding to the delayed phase (or the phase difference) (e.g., 90 degrees). When the delayed time value is, for example, Δt, the delayed phase value (e.g., 90 degrees) may be “(Δt/walking period (T))×360 degrees”. Due to this phase delay (or time delay), the wearable device 110 may change the rotation direction of a torque at the change timepoint of the rotation direction of the joint.

According to an embodiment, the filter 910 may be a digital filter and may be implemented as software. The processor 310 may execute the software implementing the filter 910 and perform filtering of the filter 910 according to the execution of the software. Embodiments are not limited thereto, and the filter 910 may be implemented, for example, as hardware, and may be included in the processor 310 or located outside the processor 310.

FIGS. 12 and 13 are diagrams illustrating examples of a signal processing operation of a wearable device according to an embodiment.

According to an embodiment, the wearable device 110 may perform a signal processing operation (e.g., a conversion operation of the converter 1210 and/or an amplification operation of the amplification unit 1220 of FIGS. 12 and 13) on the filter output y_{f}(t). The wearable device 110 may transmit a signal (e.g., an amplified converted signal Ky_{c}(t) of FIGS. 12 and 13) generated according to the performing of the signal processing operation to the driving module 30, so that a torque corresponding to the amplified converted signal Ky_{c}(t) may be generated by the driving module 30.

According to an embodiment, the wearable device 110 may include the converter 1210 for converting a filter output y_{f}(t) and/or the amplification unit 1220 for amplifying an output signal (e.g., a converted signal of FIGS. 12 and 13) of the converter 1210. As in the example shown in FIG. 12, the converter 1210 and the amplification unit 1220 may be included in the processor 310. Embodiments are not limited thereto, and as in the example shown in FIG. 13, the converter 1210 and the amplification unit 1220 may be located outside the processor 310.

According to an embodiment, the wearable device 110 (e.g., the processor 310) may generate the converted signal y(t) by converting the filter output y_{f}(t) through the converter 1210. The converter 1210 may include, for example, a digital-to-analog converter (DAC). The wearable device 110 (e.g., the processor 310) may generate the converted signal y_{c}(t), which is in the form of an analog signal, by converting the filter output y_{f}(t), which is in the form of a digital signal, through the converter 1210. The converter 1210 may transmit the converted signal y_{c}(t) to the amplification unit 1220.

According to an embodiment, the wearable device 110 (e.g., the processor 310) may amplify the converted signal y_{c}(t) through the amplification unit 1220. For example, the amplification unit 1220 may amplify the converted signal y_{c}(t) according to the value of a gain K related to the torque magnitude, and output the amplified converted signal Ky_{c}(t). The processor 310 may control the driving module 30 using the amplified converted signal Ky_{c}(t).

According to an embodiment, the amplification unit 1220 may include a first amplifier and a second amplifier. The first amplifier may perform an operation of amplifying a given input, and the second amplifier may perform an operation of inverting a given input and amplifying the inverted input. “Inverting” may refer to, for example, an operation of turning a given input A into −A. The second amplifier may correspond to an inverting amplifier.

According to an embodiment, the wearable device 110 may operate in an assistance mode. For example, the wearable device 110 may receive control information in which the operation mode is determined to be an assistance mode from the electronic device 120 and/or the other wearable device 130, and operate in the assistance mode according to the received control information. As another example, the wearable device 110 may receive an input of selecting the operation mode of the wearable device 110 to be an assistance mode from a user, and operate in the assistance mode according to the received input.

In the assistance mode, the processor 310 may determine the value of a gain K to be a value with a first sign (e.g. a positive value), and transmit the value of the gain K to the amplification unit 1220. Since the value of the gain K received from the processor 310 is a value with the first sign (e.g., a positive value), the amplification unit 1220 may amplify the converted signal y_{c}(t) using the first amplifier. For example, the value of the gain K may be a positive value K₁. In this case, the form of the output signal of the amplification unit 1220 (e.g., the first amplifier) may correspond to, for example, K₁y_{c}(t). The processor 310 may control the driving module 30 using the control signal in the form of K₁y_{c}(t).

According to an embodiment, the wearable device 110 may operate in a resistance mode. For example, the wearable device 110 may receive control information in which the operation mode is determined to be a resistance mode from the electronic device 120 and/or the other wearable device 130, and operate in the resistance mode according to the received control information. As another example, the wearable device 110 may receive an input of selecting the operation mode of the wearable device 110 to be a resistance mode from a user, and operate in the resistance mode according to the received input.

In the resistance mode, the processor 310 may determine the value of a gain K to be a value with a second sign (e.g. a negative value), and transmit the value of the gain K to the amplification unit 1220. Since the value of the gain K received from the processor 310 is a value with the second sign (e.g., a negative value), the amplification unit 1220 may invert the converted signal y(t) using the second amplifier and amplify the inverted converted signal −y_{c}(t). For example, the value of the gain K may be −K₁. In this case, the form of the output signal of the amplification unit 1220 (e.g., the second amplifier) may correspond to, for example, −K₁y_{c}(t). The processor 310 may control the driving module 30 using the control signal in the form of −K₁y_{c}(t).

Depending on the implementation, unlike the examples shown in FIGS. 12 and 13, the processor 310 may multiply the filter output y_{f}(t), which is in the form of a digital signal, by the value of the gain K. At this time, the processor 310 may determine the value of the gain K to be a value with the first sign (e.g., a positive value) when the operation mode of the wearable device 110 is an assistance mode, and determine the value of the gain K to be a value with the second sign (e.g., a negative value) when the operation mode of the wearable device 110 is a resistance mode. The processor 310 may convert the filter output Ky_{f}(t) multiplied by the value of the gain K into an analog signal through the converter 1210. The form of the output signal of the converter 1210 may correspond to, for example, Ky_{c}(t).

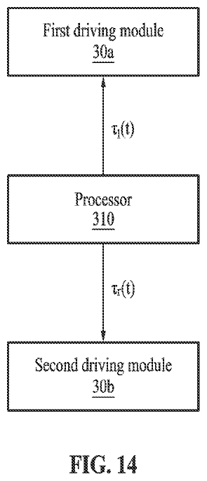

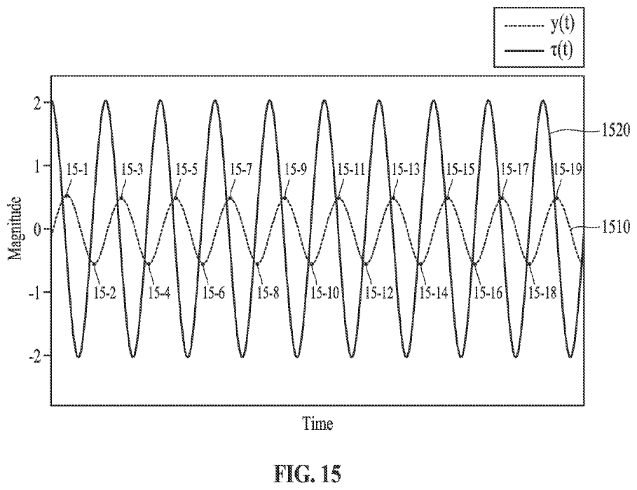

FIGS. 14 and 15 are diagrams illustrating examples of an operation of a wearable device according to an embodiment.

According to an embodiment, the processor 310 may control the driving module 30 based on a filter output y_{f}(t) so that a torque based on the filter output y_{f}(t) may be generated by the driving module 30.

For example, the processor 310 may perform an inversion operation on an amplified converted signal Ky_{c}(t), and transmit an inverted signal −Ky_{c}(t) to the first driving module 30a. The processor 310 may transmit the amplified converted signal Ky_{c}(t) to the second driving module 30b. In FIG. 14, τₗ(t) may be −Ky_{c}(t), and τᵣ(t) may be Ky_{c}(t). τₗ(t) transmitted by the processor 310 to the first driving module 30a is referred to as a first control signal, and τᵣ(t) transmitted by the processor 310 to the second driving module 30b is referred to as a second control signal.

The processor 310 may control the first driving module 30a using the first control signal τₗ(t) so that a torque corresponding to the first control signal τₗ(t) may be generated by the first driving module 30a. The first driving module 30a may generate a torque according to the first control signal τₗ(t) and provide the generated torque to the user (e.g., the left leg of the user). The processor 310 may control the second driving module 30b using the second control signal τᵣ(t) so that a torque corresponding to the second control signal τᵣ(t) may be generated by the second driving module 30b. The second driving module 30b may generate a torque according to the second control signal τᵣ(t) and provide the generated torque to the user (e.g., the right leg of the user).

According to an embodiment, the filter output y_{f}(t) may have a phase delayed by a first phase value (e.g., 90 degrees) from the phase of the filter input y(t), and the control signal τₗ(t) and/or τᵣ(t) based on the filter output y_{f}(t) may also have a phase delayed by the first phase value (e.g., 90 degrees) from the phase of the filter input y(t). As the phase of the control signal τₗ(t) and/or τᵣ(t) is delayed by the first phase value (e.g., 90 degrees) from the phase of the filter input y(t), the change timepoint of the rotation direction of a joint of the user and the change timepoint of the torque rotation direction may correspond to each other (or match) in the walking period of the user. An example of the filter input y(t) and an example of the control signal τ(t) (e.g., τₗ(t) or τᵣ(t)) are shown in FIG. 15.

In the example shown in FIG. 15, peak values 15-1 to 15-19 of a filter input y(t) 1510 may be, for example, the points at which the rotation direction of a joint of the user changes. The value of a control signal τ(t) 1520 may be “0” at the peak values 15-1 to 15-19 of the filter input y(t) 1510. The sign of the value of the control signal τ(t) 1520 may change before and after the peak values 15-1 to 15-19 of the filter input y(t) 1510. For example, the sign of the value of the control signal τ(t) 1520 may change from plus to minus before and after the positive peak values 15-1, 15-3, 15-5, 15-7, 15-9, 15-11, 15-13, 15-15, 15-17, and 15-19 of the filter input y(t) 1510. The sign of the value of the control signal τ(t) 1520 may change from minus to plus before and after the negative peak values 15-2, 15-4, 15-6, 15-8, 15-10, 15-12, 15-14, 15-16, and 15-18 of the filter input y(t) 1510. This may indicate that the torque rotation direction changes when the rotation direction of the joint of the user changes (or at the peak values of the filter input y(t) 1510.

As described above, changing the rotation direction of the torque changes when the rotation direction of the joint changes may allow the user to feel a natural torque suitable for the motion of the user. The wearable device 110 may change the torque rotation direction of the driving module 30 at the change timepoint of the rotation direction of the joint of the user (or at the peak values of the filter input y(t) 1510) through phase delay of the filter 910, thereby providing a natural torque suitable for the motion of the user to the user.

Additionally, the wearable device 110 may cause a time delay suitable for the walking speed of the user through phase delay, thereby providing a torque optimized for the walking of the user to the user. For example, the walking period of the user when the walking speed of the user is fast may be T1, and the walking period of the user when the walking speed of the user is slow may be T2. At this time, T2 may be greater than T1. When the walking period is T1 (or when the walking speed of the user is fast), the output timing of a torque may be delayed by Δt1 by phase delay of the filter 910. When the walking period is T2 (or when the walking speed of the user is slow), the output timing of a torque may be delayed by Δt2 by phase delay of the filter 910. The delayed phase value may be “(Delayed time value (Δt)/walking period (T))×360 degrees”. Both when the walking period is T1 and when the walking period is T2, the delayed phase value may be the same, and T2 may be greater than T1. Accordingly, Δt2 may also be greater than Δt1, and the output timing of the torque may be more delayed when the walking speed of the user is slow, compared to when the walking speed of the user is fast. Through phase delay, the wearable device 110 may cause a time delay (e.g., Δt1) suitable for the fast walking speed of the user, and cause a time delay (e.g., Δt2) suitable for the slow walking speed of the user.

Additionally, existing wearable devices may shift a filter output (e.g., f(t)) tx on the time axis, and output a torque based on the shifted filter output (e.g., f(t−tx)). The existing wearable devices need to use a buffer to shift the filter output (e.g., f(t)) by tx on the time axis. This is a factor that increases the burden of memory usage of the existing wearable devices. However, according to an embodiment, the wearable device 110 may only cause the filter output y_{f}(t) to have a delayed phase value (e.g., 90 degrees) through phase delay, and may not use the filter output (e.g., y_{f}(t−tx)) shifted on the time axis. The wearable device 110 may not use the buffer for shifting the filter output y_{f}(t) by tx on the time axis, thereby alleviating the burden of memory usage of the wearable device 110.

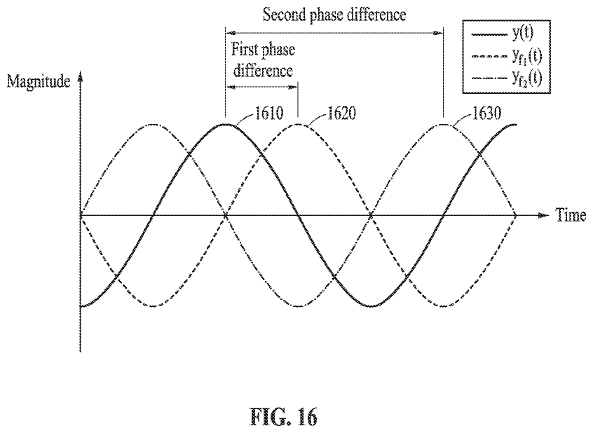

FIG. 16 is a diagram illustrating an example of an operation of a wearable device according to an embodiment.

According to an embodiment, an assistance torque and a resistance torque of the wearable device 110 may be distinguished according to the sign of the value of a gain. As described above, when the sign of the value of the gain is, for example, a first sign (e.g., plus) or the value of the gain is positive, the wearable device 110 may generate an assistance torque. When the sign of the value of the gain is, for example, a second sign (e.g., minus) or the value of the gain is negative, the wearable device 110 may generate a resistance torque. Unlike this embodiment, according to another embodiment, the assistance torque and the resistance torque of the wearable device 110 may be distinguished according to how much the phase of the filter input y(t) is delayed. In this case, the value of the gain in an assistance mode and a resistance mode may be positive.

According to an embodiment, the wearable device 110 may operate in an assistance mode. For example, the wearable device 110 may receive control information in which the operation mode is determined to be an assistance mode from the electronic device 120 and/or the other wearable device 130, and operate in the assistance mode according to the received control information. As another example, the wearable device 110 may receive an input of selecting the operation mode of the wearable device 110 to be an assistance mode from a user, and operate in the assistance mode according to the received input.

In the assistance mode, the processor 310 may user a first filter (e.g., the filter 910) for filtering a filter input y(t) 1610. The processor 310 may determine a cutoff frequency value of the first filter (e.g., the filter 910) based on a walking period of a user, and determine a filter parameter value of the first filter (e.g., the filter 910) using the determined cutoff frequency value. The processor 310 may delay the phase of the filter input y(t) 1610 by a first phase value (e.g., 90 degrees) through the first filter (e.g., the filter 910). A filter output y_{f1}(t) 1620 generated through the filtering by the first filter (e.g., the filter 910) may have a first phase difference (e.g., 90 degrees) by phase delay as in the example shown in FIG. 16. The processor 310 may generate an amplified converted signal (e.g., K₁y_{c1}(t)) by performing a signal processing operation (e.g., the conversion operation and/or the amplification operation described with reference to FIGS. 12 and 13) on the filter output y_{f1}(t) 1620. For example, if the value of the gain is K₁, a control signal to control the driving module 30 by the processor 310 in the assistance mode may be in the form of K₁y_{c1}(t).

According to an embodiment, the wearable device 110 may operate in a resistance mode. For example, the wearable device 110 may receive control information in which the operation mode is determined to be a resistance mode from the electronic device 120 and/or the other wearable device 130, and operate in the resistance mode according to the received control information. As another example, the wearable device 110 may receive an input of selecting the operation mode of the wearable device 110 to be a resistance mode from a user, and operate in the resistance mode according to the received input.

In the resistance mode, the processor 310 may use a second filter for filtering the filter input y(t) 1610. The second filter may have a phase response characteristic that delays the phase of a given input by a second phase value (e.g., 270 degrees) at a cutoff frequency value. The second filter may include, for example, a high-order low-pass filter (e.g., a high-order Butterworth low-pass filter), but is not limited thereto. Here, “high-order” may be sixth-order or higher, but is not limited thereto.

The processor 310 may determine a cutoff frequency value of the second filter based on the walking period of the user. For example, the cutoff frequency value of the second filter may be the same as the cutoff frequency value of the first filter. The processor 310 may determine a filter parameter value of the second filter (e.g., a filter coefficient of the second filter) using the cutoff frequency value of the second filter. The processor 310 may delay the phase of the filter input y(t) by the second phase value (e.g., 270 degrees) through the second filter. A filter output y_{f2}(t) 1630 generated through filtering by the second filter may have a second phase difference (e.g., 270 degrees) by phase delay as in the example shown in FIG. 16. The processor 310 may generate an amplified converted signal (e.g., Ky_{c2}(t)) by performing a signal processing operation (e.g., the conversion operation and/or the amplification operation described with reference to FIGS. 12 and 13) on the filter output y_{f2}(t) 1630. For example, if the value of the gain is K₁, a control signal to control the driving module 30 by the processor 310 in the resistance mode may be in the form of K₁y_{c2}(t).

As in the example shown in FIG. 16, the filter output y_{f2}(t) 1630 and the filter output y_{f1}(t) 1620 may have a phase difference of 180 degrees. Accordingly, the form of y_{f2}(t) 1630 may be the form of −y_{f1}(t), the form of y_{c2}(t) may be the form of −y_{c1}(t), and the form of K₁y_{c2}(t) may be the form of −K₁y_{c1}(t). The form of a control signal −K₁y_{c1}(t) based on the filter output y_{f2}(t) 1630 may be the same as the form of a control signal used to control the driving module 30 by the processor 310 when the value of the gain is negative. The wearable device 110 may delay the phase of the filter input y(t) by the second phase value (e.g., 270 degrees) through the second filter, thereby providing a resistance torque to the user.

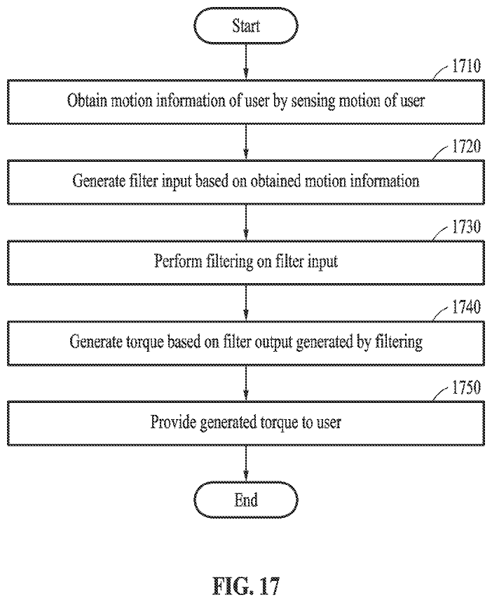

FIG. 17 is a flowchart illustrating an example of an operating method of a wearable device according to an embodiment.

Referring to FIG. 17, in operation 1710, the wearable device 110 may obtain motion information of a user by sensing a motion of the user. The motion information may include time series data (e.g., time series data with periodicity) obtained when the user performs a motion (or exercise) (e.g., walking). For example, the motion information may include first angle data obtained by measuring a first joint angle (e.g., the right hip joint angle, the right knee joint angle, etc.) of the user and/or second angle data obtained by measuring a second joint angle (e.g., the left hip joint angle, the left knee joint angle, etc.) of the user. As another example, the motion information may include first angular velocity data (or first angular acceleration data) about the angular velocity (or angular acceleration) of a first joint of the user and/or second angular velocity data (or second angular acceleration data) about the angular velocity (or angular acceleration) of a second joint of the user. As another example, the motion information may include acceleration information and/or rotation angle information of the user obtained by the IMU 360. The motion information is not limited to the examples described above.

In operation 1710, one or more sensors (e.g., at least one of the angle sensor 320, the angle sensor 320-1, or the IMU 360) of the wearable device 110 may obtain the motion information of the user by sensing (or measuring) the motion of the user, and transmit the obtained motion information to the processor 310.

In operation 1720, the wearable device 110 may generate a filter input based on the obtained motion information. The processor 310 may generate the filter input by processing the obtained motion information so that the obtained motion information may have the form of a sinusoidal wave. For example, the processor 310 may generate the filter input according to Equation 1, Equation 2, or Equation 3.

In operation 1730, the wearable device 110 may perform filtering on the filter input through the filter 910. The wearable device 110 (e.g., the processor 310) may perform filtering by delaying the phase of the filter input through the filter 910 so that the change timepoint of the torque rotation direction of the driving module 30 may correspond to the peak timepoint of the filter input.

In operation 1740, the wearable device 110 may generate a torque based on a filter output generated by filtering. The wearable device 110 (e.g., the processor 310) may perform a signal processing operation (e.g., conversion through the converter 1210 and/or amplification through the amplification unit 1220) on the generated filter output. The wearable device 110 (e.g., the processor 310) may control the driving module 30 using a signal (e.g., Ky_{c}(t)) generated according to the signal processing operation, so that a torque corresponding to the signal (e.g., Ky_{c}(t)) may be generated by the driving module 30.

In operation 1750, the wearable device 110 may provide the generated torque (e.g., an assistance torque or a resistance torque) to the user.

According to an embodiment, the wearable device 110 (e.g., the processor 310) may determine a walking period of the user using the obtained motion information. For example, the processor 310 may perform operation 810 of FIG. 8 described above. The wearable device 110 (e.g., the processor 310) may determine a cutoff frequency value of the filter 910 using the determined walking period.

According to an embodiment, the wearable device 110 (e.g., the processor 310) may determine filter parameter values (e.g., the values of a₁, a₂, b₀, b₁, and b₂) of the filter 910 using the determined cutoff frequency value. For example, the processor 310 may determine the values of a₁ and a₂ according to Equation 5, and determine the values of b₀, b₁, and b₂ according to Equation 6.

According to an embodiment, the wearable device 110 (e.g., the processor 310) may generate a filter output having a phase delayed by a first phase value (e.g., 90 degrees) from the phase of the filter input by performing filtering on the filter input through the filter 910 having the determined filter parameter values.

According to an embodiment, the wearable device 110 (e.g., the processor 310) may control the driving module 30 based on the filter output, so that the torque rotation direction may change to match the change timepoint of the joint rotation direction of the user.

According to an embodiment, the wearable device 110 (e.g., the processor 310) may generate a converted signal by converting the filter output through the converter 1210. The wearable device 110 (e.g., the processor 310) may amplify the generated converted signal through the amplification unit 1220 and control the driving module 30 based on the amplified converted signal.

The embodiments described with reference to FIGS. 1 to 16 may apply to the operating method of the wearable device 110 of FIG. 17.

According to an embodiment, a wearable device 110 may include a driving module 30 configured to generate a torque and provide the generated torque to the user, a first sensor 320 configured to measure a first joint angle of the user, a second sensor 320-1 configured to measure a second joint angle of the user, and a processor 310. The processor may generate a filter input to be filtered by a filter 910 based on a difference between first angle data obtained by measuring the first joint angle by the first sensor and second angle data obtained by measuring the second joint angle by the second sensor. The processor may perform filtering on the generated filter input through the filter so that a change timepoint of a torque rotation direction of the driving module may correspond to a peak timepoint of the generated filter input by delaying a phase of the generated filter input through the filter. The processor may control the driving module based on a filter output generated by the filtering, so that a torque based on the generated filter output may be generated by the driving module.

According to an embodiment, the processor may determine a walking period of the user using at least one of the first angle data or the second angle data. The processor may determine a cutoff frequency value of the filter using the determined walking period.

According to an embodiment, the processor may determine a filter parameter value of the filter using the determined cutoff frequency value.

According to an embodiment, the processor may generate the filter output having a phase delayed by a first phase value from the phase by performing the filtering through the filter having the determined filter parameter value.

According to an embodiment, the first phase value may include 90 degrees.

According to an embodiment, the processor may control the driving module based on the generated filter output, so that the torque rotation direction may change to match a change timepoint of a joint rotation direction of the user.

According to an embodiment, the processor may generate a converted signal by converting the generated filter output through a converter 1210. The processor may amplify the generated converted signal through an amplification unit 1220, and control the driving module based on the amplified converted signal.

According to an embodiment, the filter may include a second-order low-pass filter.

According to an embodiment, a wearable device 110 may include a driving module 30 configured to generate a torque and provide the generated torque to the user, one or more sensors (e.g., at least one of the angle sensor 320, the angle sensor 320-1, or the IMU 360) configured to obtain motion information of a user by sensing a motion of the user, and a processor 310. The processor may generate a filter input based on the obtained motion information. The processor may perform filtering on the generated filter input through a filter so that a change timepoint of a torque rotation direction of the driving module may correspond to a peak timepoint of the generated filter input by delaying a phase of the generated filter input through the filter. The processor may control the driving module based on a filter output generated by the filtering, so that a torque based on the generated filter output may be generated by the driving module.

According to an embodiment, the processor may determine a walking period of the user using the obtained motion information, and determine a cutoff frequency value of the filter using the determined walking period.

According to an embodiment, the processor may determine a filter parameter value of the filter using the determined cutoff frequency value.

According to an embodiment, the processor may generate the filter output having a phase delayed by a first phase value from the phase by performing the filtering through the filter having the determined filter parameter value.

According to an embodiment, the first phase value may include 90 degrees.

According to an embodiment, the processor may control the driving module based on the generated filter output, so that the torque rotation direction may change to match a change timepoint of a joint rotation direction of the user.

According to an embodiment, the processor may generate a converted signal by converting the generated filter output through a converter 1210, amplify the generated converted signal through an amplification unit 1220, and control the driving module based on the amplified converted signal.

According to an embodiment, the filter may include a second-order low-pass filter.

According to an embodiment, the obtained motion information may include first angle data obtained by measuring a first joint angle of the user and second angle data obtained by measuring a second joint angle of the user.

According to an embodiment, an operating method of a wearable device 110 may include measuring a first joint angle of a user and measuring a second joint angle of the user. The operating method of the wearable device 110 may include generating a filter input based on a difference between first angle data obtained by measuring the first joint angle and second angle data obtained by measuring the second joint angle. The operating method of the wearable device 110 may include performing filtering on the generated filter input through a filter so that a change timepoint of a torque rotation direction of a driving module of the wearable device may correspond to a peak timepoint of the generated filter input by delaying a phase of the generated filter input through the filter. The operating method of the wearable device 110 may include generating a torque based on a filter output generated by the filtering. The operating method of the wearable device 110 may include providing the generated torque to the user. “Based on” as used herein covers based at least on.

According to an embodiment, the operating method of the wearable device 110 may further include determining a walking period of the user using at least one of the first angle data or the second angle data, and determining a cutoff frequency value of the filter using the determined walking period.

Each embodiment herein may be used in combination with any other embodiment(s) described herein.

According to an embodiment, the operating method of the wearable device 110 may further include determining a filter parameter value of the filter using the determined cutoff frequency value. The performing of the filtering may include generating the filter output having a phase delayed by a first phase value from the phase by performing the filtering through the filter having the determined filter parameter value.

The units described herein may be implemented using a hardware component, a software component and/or a combination thereof. A processing device may be implemented using one or more general-purpose or special-purpose computers, such as, for example, a processor, a controller and an arithmetic logic unit (ALU), a digital signal processor (DSP), a microcomputer, a field-programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described hardware devices may be configured to act as one or more software modules in order to perform the operations of the above-described examples, or vice versa.

A number of embodiments have been described above. Nevertheless, it should be understood that various modifications may be made to these embodiments. For example, suitable results may be achieved if the described techniques are performed in a different order, and/or if components in a described system, architecture, device, or circuit are combined in a different manner, or replaced or supplemented by other components or their equivalents. Therefore, other implementations, other embodiments, and/or equivalents of the claims are within the scope of the following claims.

What is claimed is:

1. A wearable device comprising:
a driving module, comprising a motor and/or circuitry, configured to generate a torque and provide the generated torque to the user;
a first sensor configured to measure a first joint angle of the user;
a second sensor configured to measure a second joint angle of the user; and
at least one processor, comprising processing circuitry, individually and/or collectively configured to generate a filter input to be filtered by a filter based on a difference between first angle data obtained by measuring the first joint angle by the first sensor and second angle data obtained by measuring the second joint angle by the second sensor, perform filtering on the generated filter input via at least the filter so that a change timepoint of a torque rotation direction of the driving module corresponds to a peak timepoint of the generated filter input at least by delaying a phase of the generated filter input via the filter, and control the driving module based on a filter output generated by the filtering, so that a torque based on the generated filter output is generated by the driving module.

2. The wearable device of claim 1, wherein at least one processor, comprising processing circuitry, is individually and/or collectively configured to determine a walking period of the user based on at least one of the first angle data or the second angle data, and determine a cutoff frequency value of the filter using the determined walking period.

3. The wearable device of claim 2, wherein at least one processor, comprising processing circuitry, is individually and/or collectively configured to determine a filter parameter value of the filter based on the determined cutoff frequency value.

4. The wearable device of claim 3, wherein at least one processor, comprising processing circuitry, is individually and/or collectively configured to generate the filter output having a phase delayed by a first phase value from the phase at least by performing the filtering via the filter having the determined filter parameter value.

5. The wearable device of claim 4, wherein the first phase value comprises 90 degrees.

6. The wearable device of claim 1, wherein at least one processor, comprising processing circuitry, is individually and/or collectively configured to control the driving module based on the generated filter output, so that the torque rotation direction changes to match a change timepoint of a joint rotation direction of the user.

7. The wearable device of claim 1, wherein at least one processor, comprising processing circuitry, is individually and/or collectively configured to generate a converted signal at least by converting the generated filter output via a converter, amplify the generated converted signal via an amplification unit, and control the driving module based on the amplified converted signal.

8. The wearable device of claim 1, wherein the filter comprises a second-order low-pass filter.

9. A wearable device comprising:
a driving module, comprising a motor and/or circuitry, configured to generate a torque and provide the generated torque to the user;
one or more sensors configured to obtain motion information of a user by sensing a motion of the user; and
at least one processor, comprising processing circuitry, individually and/or collectively configured to generate a filter input based on the obtained motion information, perform filtering on the generated filter input via a filter so that a change timepoint of a torque rotation direction of the driving module corresponds to a peak timepoint of the generated filter input by delaying a phase of the generated filter input via the filter, and control the driving module based on a filter output generated by the filtering, so that a torque based on the generated filter output is generated by the driving module.

10. The wearable device of claim 9, wherein at least one processor, comprising processing circuitry, is individually and/or collectively configured to determine a walking period of the user based on the obtained motion information, and determine a cutoff frequency value of the filter based on the determined walking period.

11. The wearable device of claim 10, wherein at least one processor, comprising processing circuitry, is individually and/or collectively configured to determine a filter parameter value of the filter based on the determined cutoff frequency value.

12. The wearable device of claim 11, wherein at least one processor, comprising processing circuitry, is individually and/or collectively configured to generate the filter output having a phase delayed by a first phase value from the phase at least by performing the filtering via the filter having the determined filter parameter value.

13. The wearable device of claim 12, wherein the first phase value comprises 90 degrees.

14. The wearable device of claim 9, wherein at least one processor, comprising processing circuitry, is individually and/or collectively configured to control the driving module based on the generated filter output, so that the torque rotation direction changes to match a change timepoint of a joint rotation direction of the user.

15. The wearable device of claim 9, wherein at least one processor, comprising processing circuitry, is individually and/or collectively configured to generate a converted signal by converting the generated filter output through a converter, amplify the generated converted signal through an amplification unit, and control the driving module based on the amplified converted signal.

16. The wearable device of claim 9, wherein the filter comprises a second-order low-pass filter.

17. The wearable device of claim 9, wherein the obtained motion information comprises first angle data obtained by measuring a first joint angle of the user and second angle data obtained by measuring a second joint angle of the user.

18. An operating method of a wearable device, the operating method comprising:
measuring a first joint angle of a user and measuring a second joint angle of the user;
generating a filter input based on a difference between at least first angle data obtained by measuring the first joint angle and second angle data obtained by measuring the second joint angle;
performing filtering on the generated filter input via a filter at least by delaying a phase of the generated filter input via the filter, so that a change timepoint of a torque rotation direction of a driving module of the wearable device corresponds to a peak timepoint of the generated filter input;
generating a torque based on a filter output generated by the filtering; and
providing the generated torque to the user.

19. The operating method of claim 18, further comprising:
determining a walking period of the user using at least one of the first angle data and/or the second angle data; and
determining a cutoff frequency value of the filter using the determined walking period.

20. The operating method of claim 19, further comprising:
determining a filter parameter value of the filter using the determined cutoff frequency value, wherein
the performing of the filtering comprises generating the filter output having a phase delayed by a first phase value from the phase by performing the filtering through the filter having the determined filter parameter value.
